(19) United States
(12) Patent Application Publication
(10) Pub. No.: US 2025/0282718 A1
(43) Pub. Date: Sep. 11, 2025

(54) METHOD FOR PREPARING A FUNCTIONALISED LITHIUM SALT

(71) Applicant: Arkema France, Colombes (FR)

(72) Inventor: Nadim EID, Pierre-Benite Cedex (FR)

(21) Appl. No.: 18/857,911

(22) PCT Filed: Apr. 18, 2023
(86) PCT No.: PCT/FR2023/050550

§ 371 Date: Oct. 18, 2024

(30) Foreign Application Priority Data
Apr. 19, 2022 (FR) FR2203585

(51) Int. Cl. C07C 311/48; C08F 222/40; H01M 4/62; H01M 10/0525; H01M 50/409

(52) U.S. Cl. CPC C07C 311/48; C08F 222/408; H01M 4/621; H01M 10/0525; H01M 50/409; C07C 2531/02

(57) ABSTRACT

The present invention relates to a process for preparing a lithium salt A, comprising step a) of placing in contact a lithium salt B comprising at least one —S(O)₂—F group with a compound C of formula (I) R—O—SiR¹R²R³ in the presence of a catalyst D to form a mixture of products comprising said lithium salt A comprising at least one group —S(O)₂—OR; said catalyst D being a compound with a pKa of greater than 11 measured at 25° C. The present invention also relates to a lithium salt of formula (IIIa), (IIIb), (IIIc), (IIId), (IIIe), (IIIf), (IIIg), (IIIh), (IIIi), (IIIj), (IVa), (IVb), (IVc) or (IVd).

### TECHNICAL FIELD OF THE INVENTION

The present invention relates to a process for preparing a lithium salt. In particular, the present invention relates to a process for preparing a lithium salt bearing functional groups.

### Technological Background of the Invention

Li-ion batteries are used as an energy source in many devices, such as cell phones, computers and electric vehicles. Most current batteries contain a liquid solution of an electrolyte consisting of a lithium salt in a solvent, the latter generally being highly flammable (for example, carbonate-type solvents). Other emerging technologies use an electrolyte in the form of a gel, which limits the flammable liquid content without, however, being risk-free. The most promising technology is based on all-solid batteries containing no flammable chemical compounds, and offering high energy density.

Many lithium salts are sold for this type of battery application. Mention may notably be made of lithium perchlorate, lithium hexafluorophosphate, bis(trifluoromethanesulfonyl)imide and lithium bis(fluorosulfonyl)imide. Lithium sulfonyl imide salts are the most promising in terms of stability and conductivity. New lithium salts suitable for new battery technologies have been evaluated. In this respect, the use of polymer materials containing lithium sulfonyl imide salts is under consideration. For example, WO 2016/012670 describes the preparation of conductive polymers of the polyaryl ether ketone or polyether sulfone type containing lithium salts of grafted bis(sulfonyl)imides. The synthesis of this type of polymer is complex and requires numerous reaction steps, sometimes under more severe operating conditions.

There is thus a need to prepare new lithium bis(sulfonyl)imide salts in a simple procedure with good yields.

The present invention is directed toward at least partly overcoming the drawbacks of the prior art.

### SUMMARY OF THE INVENTION

According to a first aspect, the present invention provides a process for preparing a lithium salt A, comprising a step a) of placing in contact a lithium salt B comprising at least one —S(O)₂—F group with a compound C of formula (I) R—O—SiR¹R²R³ in the presence of a catalyst D to form a mixture of products comprising said lithium salt A comprising at least one group —S(O)₂—OR; said catalyst D being a compound with a pKa of greater than 11 measured at 25° C.

The present invention allows the preparation of a functionalized lithium salt in mild operating reactions with good yields and thus avoiding side reactions. In particular, the present process avoids the use of nucleophilic compounds such as alkoxides or amines, which lead to dissociation of the sulfonyl imide structure. The present process allows the fluorine atom borne by the sulfur atom of lithium salt B to be substituted with a functional group —OR derived from compound C. Said lithium salt A thus formed comprises a functional group —OR bonded to said sulfur atom. According to a preferred embodiment, the substituent R of said compound C comprises at least one carbon-carbon double bond. The presence of a carbon-carbon double bond favors the preparation of a polymer containing the lithium salt.

According to a preferred embodiment, said lithium salt B is of formula LiN(S(O)₂—F)(S(O)₂R′) and said lithium salt A is of formula (IV) LiN(S(O)₂—OR)(S(O)₂R′) in which R′ is a substituent selected from the group consisting of F, C₁-C₄ alkyl, C₁-C₄ perfluoroalkyl, C₁-C₄ alkoxy and C₁-C₄ perfluoroalkoxy; preferably C₁-C₄ alkyl, C₁-C₄ perfluoroalkyl, C₁-C₄ alkoxy and C₁-C₄ perfluoroalkoxy.

According to a preferred embodiment, said lithium salt B comprises two —S(O)₂—F groups to form a lithium salt A comprising two groups —S(O)₂—OR. Thus, said lithium salt A includes two groups —OR each including at least one carbon-carbon double bond allowing the facilitated formation of a polymer containing a lithium salt.

According to a preferred embodiment, said lithium salt B is Li—N(S(O)₂—F)₂ and said lithium salt A is of formula (III) Li—N(S(O)₂—OR)₂.

According to a preferred embodiment, said compound C is of formula (I) R—O—SiR¹R²R³ in which the groups R¹, R² and R³ are, independently of each other, selected from the group consisting of H and C₁-C₁₀ alkyl.

According to a preferred embodiment, said compound C is of formula (I) R—O—SiR¹R²R³ in which the group R is of formula R⁴—X—(Y)ₙ— in which R⁴ is a radical comprising at least one carbon-carbon double bond; X is a heteroatom selected from the group consisting of O, S and N(R⁵) in which R⁵ is H or C₁-C₁₀ alkyl; Y is a group selected from the group consisting of C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹) C(R⁸)(R⁹)]— with R⁶ and R⁷ independently selected for each carbon atom and for each unit Y from H or F, R⁸ and R⁹ independently selected for each carbon atom and for each unit Y from H, F or CH₃, m an integer from 1 to 50, z=0 if X is O; n is an integer from 1 to 50.

According to a preferred embodiment, the group R⁴ is of formula (Ia), (Ib), (Ic) or (Id):

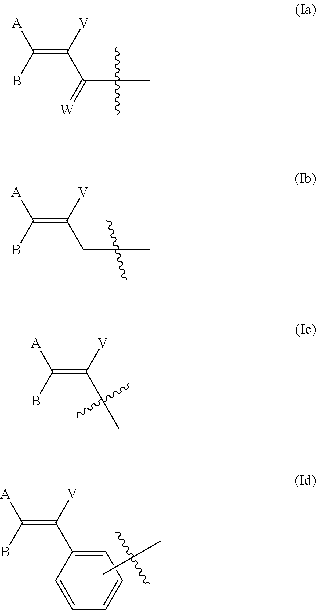

in which:
A, B and V are, independently of each other, selected from the group consisting of H, F, Cl, Br, I, C₁-C₁₀ alkyl optionally substituted with one or more substituents R″, C₂-C₁₀ alkenyl optionally substituted with one or more substituents R″, C₃-C₁₀ cycloalkyl optionally substituted with one or more substituents R″ or one or more C₁-C₅ alkyl groups, C₄-C₁₀ cycloalkenyl optionally substituted with one or more substituents R″ or one or more C₁-C₅ alkyl groups, C₆-C₁₂ aryl optionally substituted with one or more substituents R″ or one or more C₁-C₅ alkyl groups; W is selected from O and S;
R″ is selected from the group consisting of F, Cl, I, Br, C₁-C₄ ether, C₁-C₄ ester, C₁-C₄ thioether and C₁-C₄ thioester.

According to a preferred embodiment, said catalyst D is selected from the group consisting of N,N-diisopropylethylamine, 1,8-diazabicyclo(5.4.0)undec-7-ene, 6-(dibutylamino)-1,8-diazabicyclo[5.4.0]undec-7-ene, polystyrene-bound 1,8-diazabicyclo[5.4.0]undec-7-ene, 1,5,7-triazabicyclo[4.4.0]dec-5-ene, 7-methyl-1,5,7-triazabicyclo[4.4.0]dec-5-ene, 1,1,3,3-tetramethylguanidine, 2-tert-butyl-1,1,3,3-tetramethylguanidine, polystyrene-bound 1,5,7-triazabicyclo[4.4.0]dec-5-ene, 1,4-diazabicyclo[2.2.2]octane, quinuclidine, 1,5-diazabicyclo(4.3.0)non-5-ene, 2,6-di-tert-butylpyridine, 2,8,9-trimethyl-2,5,8,9-tetraaza-1-phosphabicyclo[3.3.3]undecane, cyclodiphosphazane, lithium diisopropylamide, sodium diisopropylamide, potassium diisopropylamide, magnesium diisopropylamide, calcium diisopropylamide, rubidium diisopropylamide, lithium bis(trimethylsilyl)amide, sodium bis(trimethylsilyl)amide, potassium bis(trimethylsilyl)amide, magnesium bis(trimethylsilyl)amide, calcium bis(trimethylsilyl)amide, rubidium bis(trimethylsilyl)amide, lithium tetramethylpiperidide, sodium tetramethylpiperidide, potassium tetramethylpiperidide, magnesium tetramethylpiperidide, calcium tetramethylpiperidide, rubidium tetramethylpiperidide, [18-crown-6]-KHF₂, KHF₂, N,N′-diisopropylimidazonium, bifluoride, tetrabutylammonium.

According to a preferred embodiment, said process also comprises a step b) of purifying said lithium salt A; said step b) comprising extraction of said lithium salt A with a nonpolar organic solvent.

According to a preferred embodiment, step a) is performed at a temperature of from 10° C. to 50° C.

According to a second aspect, the present invention provides a lithium salt of formula (IIIa), (IIIb), (IIIc), (IIId), (IIIe), (IIIf), (IIIg), (IIIh), (IIIi), (IIIj), (IVa), (IVb), (IVc) or (IVd)

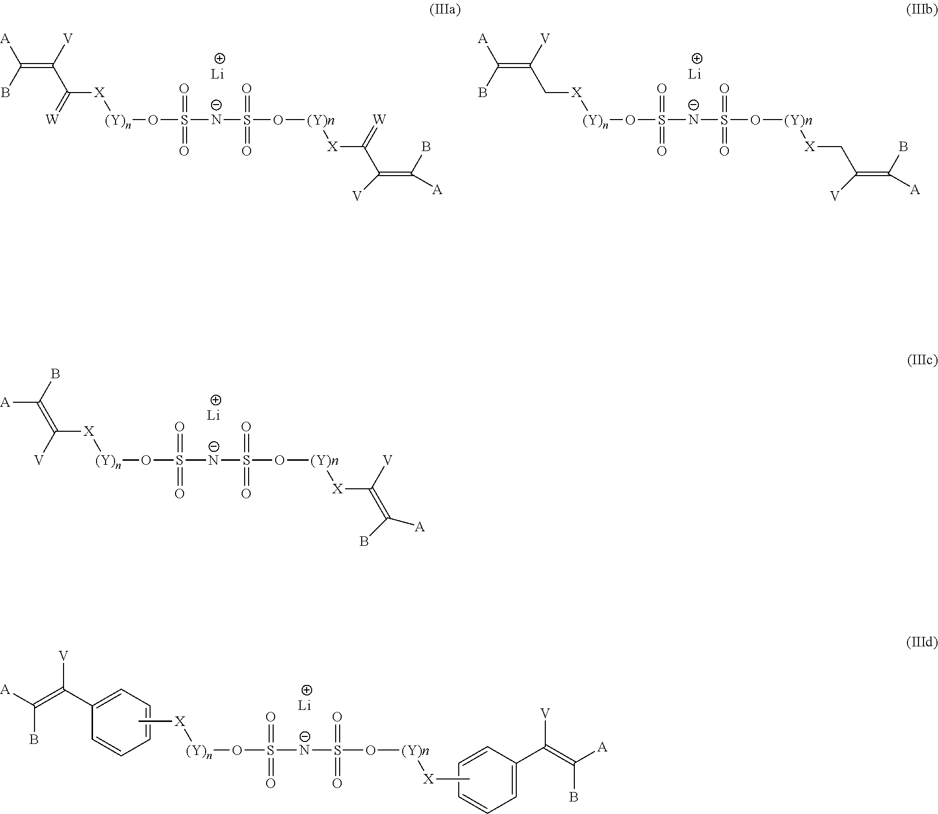

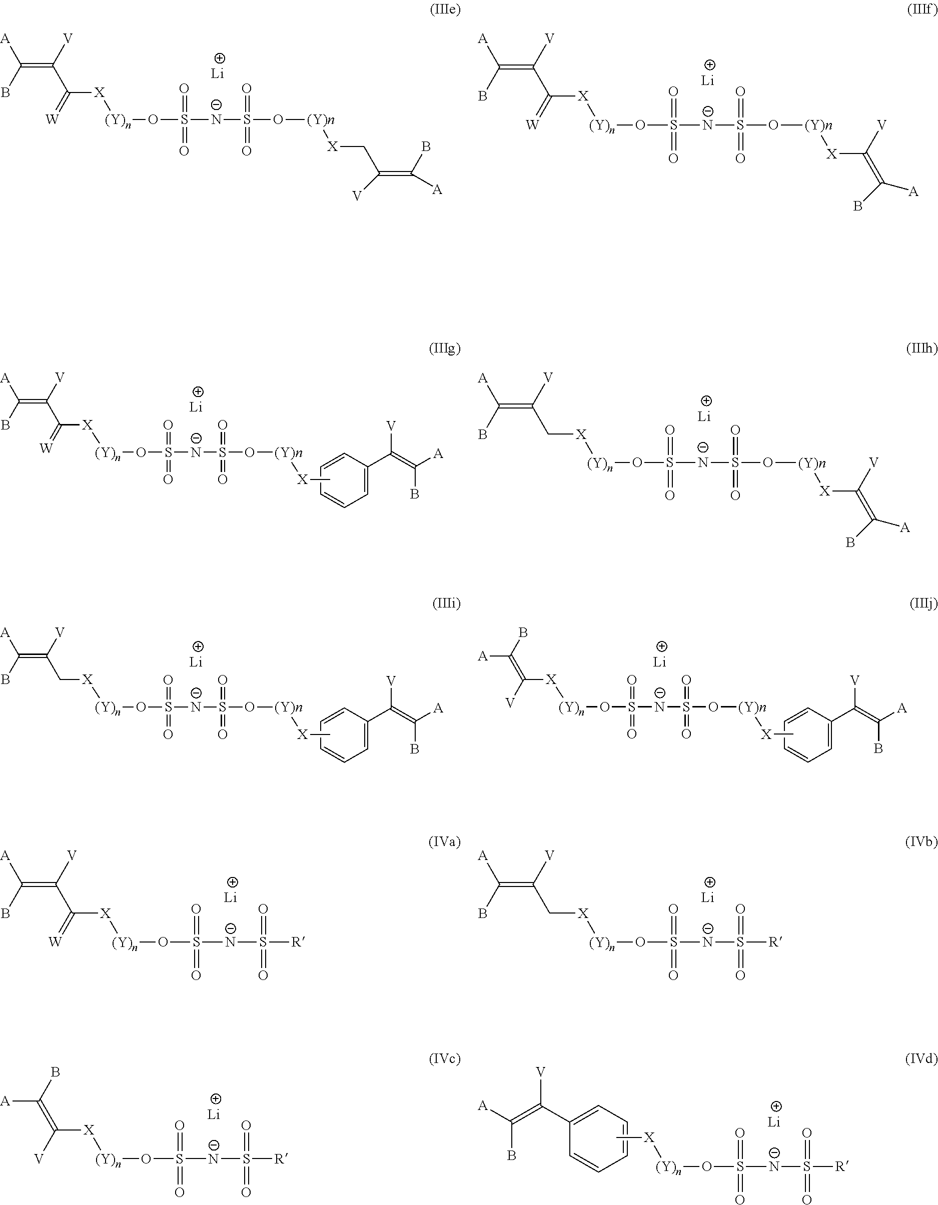

in which the substituents A, B, V and W are as defined above according to the present invention; the substituents X, Y are as defined above according to the present invention; n is as defined above according to the present invention and R′ is as defined above according to the present invention.

According to a preferred embodiment, said lithium salt is of formula (IIIa), (IIIb), (IIIc), (IIId), (IIIe), (IIIf), (IIIg), (IIIh), (IIIi), (IIIj), (IVa), (IVb), (IVc) or (IVd) in which
A and B are identical and are selected from the group consisting of H, F, Cl, Br and I;
V is selected from the group consisting of H, F, Cl, Br, I, C₁-C₁₀ alkyl optionally substituted with one or more substituents R″, C₂-C₁₀ alkenyl optionally substituted with one or more substituents R″, C₃-C₁₀ cycloalkyl optionally substituted with one or more substituents R″ or one or more C₁-C₅ alkyl groups, C₄-C₁₀ cycloalkenyl optionally substituted with one or more substituents R″ or one or more C₁-C₅ alkyl groups, C₆-C₁₂ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester; preferably, V is selected from the group consisting of H, F, Cl, Br, I, C₁-C₅ alkyl C₁-C₅ perfluoroalkyl, C₆R¹⁰₆ with R¹⁰ independently selected from H, F and C₁-C₅ alkyl;
W is O;
X is O;
Y is a group selected from the group consisting of CH₂CH₂, CH₂CF₂, CF₂CF₂, CH₂CHF, CF₂CH₂ and
CHFCH₂, and n is an integer from 1 to 25; or
Y is a group —[(C(R⁸)(R⁹)—C(R⁸)(R⁹)O)ₘ—C(R⁸)(R⁹)C(R⁸)(R⁹)]— with R⁸ and R⁹ independently selected for each carbon atom and for each unit Y from H, F or CH₃, m is an integer from 1 to 25 and n is 1;
R′ is a substituent selected from the group consisting of F, C₁-C₄ alkyl, C₁-C₄ perfluoroalkyl, C₁-C₄ alkoxy and C₁-C₄ perfluoroalkoxy; preferably C₁-C₄ alkyl, C₁-C₄ perfluoroalkyl, C₁-C₄ alkoxy and C₁-C₄ perfluoroalkoxy.

According to a preferred embodiment, said lithium salt is of formula (IIIa), (IIIb), (IIIc), (IIId), (IIIe), (IIIf), (IIIg), (IIIh), (IIIi), (IIIj), (IVa), (IVb), (IVc) or (IVd) in which A and B are H, V is CH₃, W is O, X is O and Y is a group selected from the group consisting of CH₂CH₂, CH₂CF₂, CF₂CF₂, CH₂CHF, CF₂CH₂ and CHFCH₂, and n is an integer from 1 to 10; or
Y is a group —[(C(R⁸)(R⁹)—C(R⁸)(R⁹)O)ₘ—C(R⁸)(R⁹)C(R⁸)(R⁹)]— with R⁸ and R⁹ independently selected for each carbon atom and for each unit Y from H, F or CH₃, m is an integer from 1 to 25 and n is 1;
R′ is a substituent selected from the group consisting of F, C₁-C₄ alkyl, C₁-C₄ perfluoroalkyl, C₁-C₄ alkoxy and C₁-C₄ perfluoroalkoxy; preferably C₁-C₄ alkyl, C₁-C₄ perfluoroalkyl, C₁-C₄ alkoxy and C₁-C₄ perfluoroalkoxy.

According to a preferred embodiment, said lithium salt is of formula (IIIa) in which A and B are H, V is CH₃, W is O, X is O and Y is a group selected from the group consisting of CH₂CH₂, CH₂CF₂, CF₂CF₂, CH₂CHF, CF₂CH₂ and CHFCH₂, and n is an integer from 1 to 10; or
Y is a group —[(C(R⁸)(R⁹)—C(R⁸)(R⁹)O)ₘ—C(R⁸)(R⁹)C(R⁸)(R⁹)]— with R⁸ and R⁹ independently selected for each carbon atom and for each unit Y from H, F or CH₃, m is an integer from 1 to 25 and n is 1.

According to another aspect, the present invention provides a polymer comprising monomer units derived from at least one lithium salt of formula (IIIa), (IIIb), (IIIc), (IIId), (IIIe), (IIIf), (IIIg), (IIIh), (IIIi), (IIIj), (IVa), (IVb), (IVc) or (IVd) according to the present invention.

According to a preferred embodiment, said polymer also comprises monomer units resulting from a monomer M1 of formula R¹R²C═C(R³)C(O)R in which the substituents R¹, R² and R³ are selected, independently of each other, from the group consisting of H and C₁-C₅ alkyl; R is selected from the group consisting of —NHC(CH₃)₂CH₂C(O)CH₃ or —OR′ with R′ selected from the group consisting of H and C₁-C₁₈ alkyl optionally substituted with one or more —OH groups or a 5- or 10-membered heterocycle comprising at least one nitrogen atom in its cyclic chain.

According to a preferred embodiment, said polymer also comprises monomer units resulting from a monomer M2 selected from the group consisting of vinylidene fluoride; vinyl fluoride, trifluoroethylene, chlorotrifluoroethylene, 1,2-difluoroethylene, tetrafluoroethylene, hexafluoropropylene, perfluoro(alkyl vinyl) ethers, perfluoro(1,3-dioxole), perfluoro(2,2-dimethyl-1,3-dioxole), the monomer of formula CF₂═CFOCF₂CF(CF₃)OCF₂CF₂X in which X is SO₂F, CO₂H, CH₂OH, CH₂OCN or CH₂OPO₃H, the monomer of formula CF₂═CFOCF₂CF₂SO₂F, the monomer of formula F(CF₂)nCH₂OCF═CF₂ in which n is 1, 2, 3, 4 or 5, the monomer of formula R¹CH₂OCF═CF₂ in which R¹ is hydrogen or F(CF₂)ₘ and m has the value 1, 2, 3 or 4, the monomer of formula R²OCF═CH₂ in which R² is F(CF₂)ₚ and p is 1, 2, 3 or 4, perfluorobutylethylene, trifluoropropene, tetrafluoropropene, hexafluoroisobutylene, perfluorobutylethylene, pentafluoropropene, bromotrifluoroethylene, chlorofluoroethylene, chlorotrifluoropropene or 2-trifluoromethyl-3,3,3-trifluoro-1-propene.

According to a preferred embodiment, the present invention provides a binder for an electrode or a coating for a separator comprising said polymer according to the present invention.

According to a preferred embodiment, the present invention provides an electrode or a separator comprising said polymer according to the present invention.

According to a preferred embodiment, the present invention provides a battery, preferably a lithium-ion battery, comprising said electrode or said separator according to the present invention.

### DETAILED DESCRIPTION OF THE INVENTION

According to a first aspect of the invention, a process for preparing a lithium salt A is provided. Said process comprises a step of placing in contact a lithium salt B comprising at least one —S(O)₂—F group with a compound C of formula (I) R—O—SiR¹R²R³. Said lithium salt A is formed by an exchange reaction between the fluorine atom borne by the group —S(O)₂ of said lithium salt B and the group —OR derived from compound C and borne by the silicon atom thereof. Said lithium salt A thus formed includes at least one group —S(O)₂—OR. Said process according to the present invention also results in the formation of a coproduct F—SiR¹R²R³. The present process avoids the formation of HF (the fluorine atom bonding to the Si atom) during the reaction, which would have necessitated its neutralization with an excess of base or additional purification steps. The present invention allows the preparation of a functionalized lithium salt under mild operating conditions with good yields and thus avoiding side reactions.

### Compound B

As mentioned above, said lithium salt B comprises at least one —S(O)₂—F group. According to a preferred embodiment, said lithium salt B is of formula LiN(S(O)₂—F)(S(O)₂R′) in which R′ is a substituent selected from the group consisting of F, C₁-C₄ alkyl, C₁-C₄ perfluoroalkyl, C₁-C₄ alkoxy and C₁-C₄ perfluoroalkoxy; preferably C₁-C₄ alkyl, C₁-C₄ perfluoroalkyl, C₁-C₄ alkoxy and C₁-C₄ perfluoroalkoxy. Thus, preferably, the present process comprises a step of placing LiN(S(O)₂—F)(S(O)₂R′) in contact with a compound C of formula (I) R—O—SiR¹R²R³ as defined in the present patent application, preferably in the presence of catalyst D as defined in the present patent application.

Preferably, said lithium salt B comprises two —S(O)₂—F groups.

In particular, said lithium salt B is Li—N(S(O)₂—F)₂, i.e. lithium bis(sulfonyl)imide also referred to as LiFSI. Thus, preferably, the present process comprises a step of placing in contact Li—N(S(O)₂—F)₂ with a compound C of formula (I) R—O—SiR¹R²R³ as defined in the present patent application, preferably in the presence of catalyst D as defined in the present patent application.

### Compound C

According to a preferred embodiment, said compound C is of formula (I) R—O—SiR¹R²R³.

According to a preferred embodiment, said compound C is of formula (I) R—O—SiR¹R²R³ in which the groups R¹, R² and R³ are, independently of each other, selected from the group consisting of H, C₁-C₂₀ alkyl, C₃-C₂₀ cycloalkyl and C₆-C₂₀ aryl. Preferably, the groups R¹, R² and R³ are selected, independently of each other, from the group consisting of H, C₁-C₁₅ alkyl, C₃-C₁₅ cycloalkyl and C₆-C₁₅aryl. More preferentially, the groups R¹, R² and R³ are selected, independently of each other, from the group consisting of H, C₁-C₁₀ alkyl, C₃-C₁₀ cycloalkyl and C₆-C₁₂ aryl. In particular, the groups R¹, R² and R³ are selected, independently of each other, from the group consisting of H and C₁-C₁₀ alkyl. More particularly, the groups R¹, R² and R³ are, independently of each other, selected from the group consisting of H, methyl, ethyl, propyl, isopropyl, butyl, isobutyl, tert-butyl, sec-butyl and pentyl. In a preferred manner, the groups R¹, R² and R³ are, independently of each other, selected from the group consisting of H, methyl, ethyl, propyl and tert-butyl. In a preferentially preferred manner, the groups R¹ and R² are identical and are selected from the group consisting of H, methyl and ethyl, and the group R³ is selected from the group consisting of H, methyl, ethyl, propyl and tert-butyl. In a particularly preferred manner, the groups R¹, R² and R³ are identical and are selected from the group consisting of H, methyl and ethyl.

According to a preferred embodiment, the substituent R of said compound C comprises at least one carbon-carbon double bond. As mentioned above, the presence of a carbon-carbon double bond favors the preparation of a polymer containing the lithium salt.

Preferably, the group R of said compound C is of formula R⁴—X—(Y)ₙ— in which
R⁴ is a radical comprising at least one carbon-carbon double bond;
X is a heteroatom selected from the group consisting of O, S and N(R⁵) in which R⁵ is H or C₁-C₁₀ alkyl; preferably, R⁵ is H, CH₃ or C₂H₅;
Y is a group selected from the group consisting of —C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹)C(R⁸)(R⁹)]— with
R⁶ and R⁷ independently selected for each carbon atom and for each unit Y from H or F; R⁸ and
R⁹ independently selected for each carbon atom and for each unit Y from H, F or CH₃,
m is an integer ranging from 1 to 50, advantageously from 1 to 40, preferably from 1 to 30, more preferentially from 1 to 20 and in particular from 1 to 10.
z=0 if X is O;
n is an integer ranging from 1 to 50, advantageously from 1 to 40, preferably from 1 to 30, more preferentially from 1 to 20 and in particular from 1 to 10.

According to a preferred embodiment, in said compound C, the group R⁴ is of formula (Ia), (Ib), (Ic) or (Id):

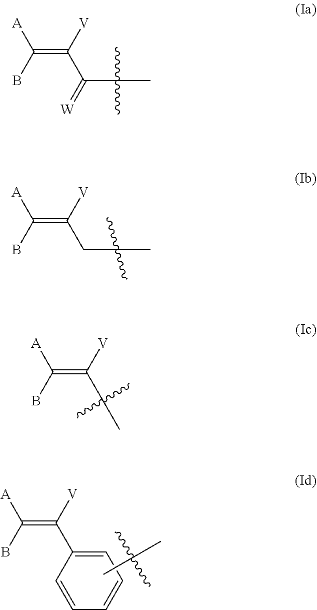

in which:
A, B and V are, independently of each other, selected from the group consisting of H, F, Cl, Br, I, C₁-C₂₀ alkyl optionally substituted with one or more substituents R″, C₂-C₂₀ alkenyl optionally substituted with one or more substituents R″, C₃-C₂₀ cycloalkyl optionally substituted with one or more substituents R″ or one or more C₁-C₅ alkyl groups, C₄-C₂₀ cycloalkenyl optionally substituted with one or more substituents R″ or one or more C₁-C₅ alkyl groups, C₆-C₂₀ aryl optionally substituted with one or more substituents R″ or one or more C₁-C₅ alkyl groups;
W is selected from O and S, preferably O;
R″ is selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester. Preferably, in said compound C, the group R⁴ is of formula (Ia), (Ib), (Ic) or (Id) as illustrated above in which A, B and V are, independently of each other, selected from the group consisting of H, F, Cl, Br, I, C₁-C₁₅ alkyl optionally substituted with one or more substituents R″, C₂-C₁₅ alkenyl optionally substituted with one or more substituents R″, C₃-C₁₅ cycloalkyl optionally substituted with one or more substituents R″ or one or more C₁-C₅ alkyl groups, C₄-C₁₅ cycloalkenyl optionally substituted with one or more substituents R″ or one or more C₁-C₅ alkyl groups, C₆-C₁₅ aryl optionally substituted with one or more substituents R″ or one or more C₁-C₅ alkyl groups;
W is selected from O and S, preferably O;
R″ is selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester.

More preferentially, in said compound C, the group R⁴ is of formula (Ia), (Ib), (Ic) or (Id) as illustrated above in which A, B and V are, independently of each other, selected from the group consisting of H, F, Cl, Br, I, C₁-C₁₀ alkyl optionally substituted with one or more substituents R″, C₂-C₁₀ alkenyl optionally substituted with one or more substituents R″, C₃-C₁₀ cycloalkyl optionally substituted with one or more substituents R″ or one or more C₁-C₅ alkyl groups, C₄-C₁₀ cycloalkenyl optionally substituted with one or more substituents R″ or one or more C₁-C₅ alkyl groups, C₆-C₁₀ aryl optionally substituted with one or more substituents R″ or one or more C₁-C₅ alkyl groups;
W is selected from O and S, preferably O;
R″ is selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester. In particular, in said compound C, the group R⁴ is of formula (Ia), (Ib), (Ic) or (Id) as illustrated above in which A, B and V are, independently of each other, selected from the group consisting of H, F, Cl, Br, I, C₁-C₁₀ alkyl, C₂-C₁₀ alkenyl, C₃-C₁₀ cycloalkyl, C₄-C₁₀ cycloalkenyl, C₆-C₁₀ aryl, C₁-C₁₀ perfluoroalkyl, C₂-C₁₀ perfluoroalkenyl, C₃-C₁₀ perfluorocycloalkyl, C₄-C₁₀ perfluorocycloalkenyl and C₆R¹⁰₅ with R¹⁰ independently selected from H, F and C₁-C₅ alkyl;
W is selected from O and S, preferably O.

More particularly, in said compound C, the group R⁴ is of formula (Ia), (Ib), (Ic) or (Id) as illustrated above in which A, B and V are, independently of each other, selected from the group consisting of H, F, Cl, Br, I, C₁-C₅ alkyl, C₁-C₅ perfluoroalkyl and C₆R¹⁰₅ with R¹⁰ independently selected from H, F and C₁-C₅ alkyl;
W is selected from O and S, preferably O.

Preferably, in said compound C, the group R⁴ is of formula (Ia), (Ib), (Ic) or (Id) as illustrated above in which A and B are, independently of each other, selected from the group consisting of H, F, Cl, I, Br, CH₃, C₂H₅, CF₃, C₂F₅ and phenyl; and V is selected from the group consisting of H, F, Cl, Br, I, C₁-C₅ alkyl, C₁-C₅ perfluoroalkyl and C₆R¹⁰₅ with R¹⁰ independently selected from H, F and C₁-C₅ alkyl;
W is selected from O and S, preferably O.

In a preferentially preferred manner, the group R⁴ is of formula (Ia), (Ib), (Ic) or (Id) as illustrated above in which
A and B are identical and are selected from the group consisting of H, F, Cl, I and Br; or
A is H and B is selected from the group consisting of F, Cl, Br, I, CH₃, C₂H₅, CF₃, C₂F₅ and phenyl.

V is selected from the group consisting of H, F, Cl, Br, I, C₁-C₅ alkyl, C₁-C₅ perfluoroalkyl and CR¹⁰₅ with R¹⁰ independently selected from H, F and C₁-C₅ alkyl;
W is selected from O and S, preferably O.

Thus, according to the present invention, said compound C may be of formula (Ia), (Ib), (Ic) or (Id)

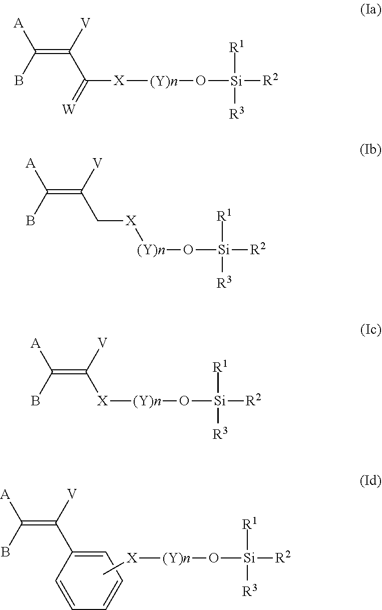

in which the substituents A, B, V, W, X, Y, R¹, R² and R³ are as defined above in the present patent application according to any one of the embodiments described.

According to a preferred embodiment, said compound C may be of formula (Ia), (Ib), (Ic) or (Id)

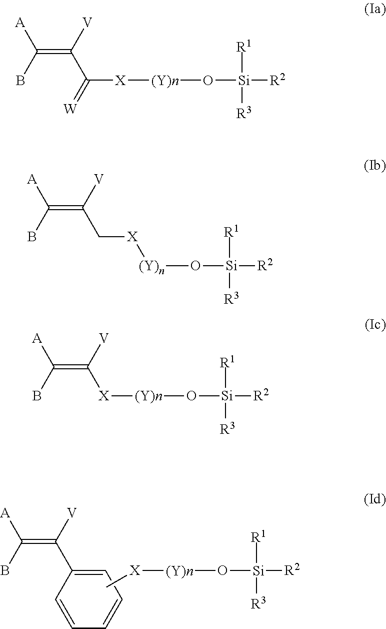

in which:
R¹, R² and R³ are, independently of each other, selected from the group consisting of H, C₁-C₂₀ alkyl, C₃-C₂₀ cycloalkyl and C₆-C₂₀ aryl; advantageously, R¹, R² and R³ are, independently of each other, selected from the group consisting of H, C₁-C₁₅ alkyl, C₃-C₁₅ cycloalkyl and C₆-C₁₅ aryl; preferably, R¹, R² and R³ are, independently of each other, selected from the group consisting of H, C₁-C₁₀ alkyl, C₃-C₁₀ cycloalkyl and C₆-C₁₂ aryl; more preferentially, R¹, R² and R³ are, independently of each other, selected from the group consisting of H and C₁-C₁₀ alkyl; in particular, R¹, R² and R³ are, independently of each other, selected from the group consisting of H, methyl, ethyl, propyl, isopropyl, butyl, isobutyl, tert-butyl, sec-butyl and pentyl; more particularly, R¹, R² and R³ are, independently of each other, selected from the group consisting of H, methyl, ethyl, propyl and tert-butyl;
X is a heteroatom selected from the group consisting of O, S and N(R⁵) in which R⁵ is H or C₁-C₁₀ alkyl;
Y is a group selected from the group consisting of —C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹)C(R⁸)(R⁹)]— with R⁶ and R⁷ independently selected for each carbon atom and for each unit Y from H or F; R⁸ and R⁹ independently selected for each carbon atom and for each unit Y from H, F or CH₃;
m is an integer ranging from 1 to 50, advantageously from 1 to 40, preferably from 1 to 30, more preferentially from 1 to 20 and in particular from 1 to 10;
z=0 if X is O;
n is an integer ranging from 1 to 50, advantageously from 1 to 40, preferably from 1 to 30, more preferentially from 1 to 20 and in particular from 1 to 10;
A, B and V are, independently of each other, selected from the group consisting of H, F, Cl, Br, I, C₁-C₂₀ alkyl optionally substituted with one or more substituents R″, C₂-C₂₀ alkenyl optionally substituted with one or more substituents R″, C₃-C₂₀ cycloalkyl optionally substituted with one or more substituents R″ or one or more C₁-C₅ alkyl groups, C₄-C₂₀ cycloalkenyl optionally substituted with one or more substituents R″ or one or more C₁-C₅ alkyl groups, C₆-C₂₀ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester; advantageously, A, B and V are, independently of each other, selected from the group consisting of H, F, Cl, Br, I, C₁-C₁₅ alkyl optionally substituted with one or more substituents R″, C₂-C₁₅ alkenyl optionally substituted with one or more substituents R″, C₃-C₁₅ cycloalkyl optionally substituted with one or more substituents R″ or one or more C₁-C₅ alkyl groups, C₄-C₁₅ cycloalkenyl optionally substituted with one or more substituents R″ or one or more C₁-C₅ alkyl groups, C₆-C₁₅ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably, F, C₁-C₄ ether, C₁-C₄ ester, C₁-C₄ thioether and C₁-C₄ thioester; preferably, A, B and V are, independently of each other, selected from the group consisting of H, F, Cl, Br, I, C₁-C₁₀ alkyl optionally substituted with one or more substituents R″, C₂-C₁₀ alkenyl optionally substituted with one or more substituents R″, C₃-C₁₀ cycloalkyl optionally substituted with one or more substituents R″ or one or more C₁-C₅ alkyl groups, C₄-C₁₀ cycloalkenyl optionally substituted with one or more substituents R″ or one or more C₁-C₅ alkyl groups, C₆-C₁₀ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester; more preferentially, A, B and V are, independently of each other, selected from the group consisting of H, F, Cl, Br, I, C₁-C₁₀ alkyl, C₂-C₁₀ alkenyl, C₃-C₁₀ cycloalkyl, C₄-C₁₀ cycloalkenyl, C₆-C₁₀ aryl, C₁-C₁₀ perfluoroalkyl, C₂-C₁₀ perfluoroalkenyl, C₃-C₁₀ perfluorocycloalkyl, C₄-C₁ perfluorocycloalkenyl and C₆-C₁ perfluoroaryl; in particular, A, B and V are, independently of each other, selected from the group consisting of H, F, Cl, Br, I, C₁-C₅ alkyl, C₁-C₅ perfluoroalkyl and C₆R¹⁰₅, with R¹⁰ independently selected from H, F and C₁-C₅ alkyl;
W is selected from O and S, preferably O.

According to a particular embodiment, said compound C, may be of formula (Ia), (Ib), (Ic) or (Id):

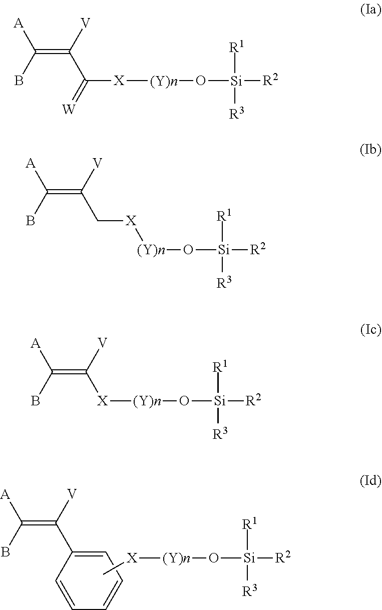

in which:
R¹ and R² are identical and are selected from the group consisting of H, methyl and ethyl, and R³ is selected from the group consisting of H, methyl, ethyl, propyl and tert-butyl; preferably, R¹, R² and R³ are identical and are selected from the group consisting of H, methyl and ethyl;
X is a heteroatom selected from the group consisting of O, S and N(R⁵) in which R⁵ is H or C₁-C₁₀ alkyl; preferably, R⁵ is H, CH₃ or C₂H₅;
Y is a group selected from the group consisting of —C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹)C(R⁸)(R⁹)]— with R⁶ and R⁷ independently selected for each carbon atom and for each unit Y from H or F; R⁸ and R⁹ independently selected for each carbon atom and for each unit Y from H, F or CH₃;
m is an integer ranging from 1 to 50, advantageously from 1 to 40, preferably from 1 to 30, more preferentially from 1 to 20 and in particular from 1 to 10;
z=0 if X is O;
n is an integer ranging from 1 to 50, advantageously from 1 to 40, preferably from 1 to 30, more preferentially from 1 to 20 and in particular from 1 to 10;
A and B are, independently of each other, selected from the group consisting of H, F, Cl, I, Br, CH₃, C₂H₅, CF₃, C₂F₅ and phenyl; preferably, A and B are identical and are selected from the group consisting of H, F, Cl, I and Br, or A is H and B is selected from the group consisting of F, Cl, Br, I, CH₃, C₂H₅, CF₃, C₂F₅ and phenyl;
V is selected from the group consisting of H, F, Cl, Br, I, C₁-C₅ alkyl, C₁-C₅ perfluoroalkyl and CR¹⁰₅ with R¹⁰ independently selected from H, F and C₁-C₅ alkyl;
W is selected from O and S, preferably O.

### Compound A

As mentioned above, said lithium salt A is formed by an exchange reaction between the fluorine atom of the S(O)₂F group borne by said lithium salt B and said group —OR derived from said compound C of formula (I) RO—SiR¹R²R³.

According to a preferred embodiment, said lithium salt A comprises at least one group —S(O)₂—OR. Preferably, said lithium salt A comprises two groups —S(O)₂—OR when said lithium salt B comprises two —S(O)₂—F groups. Thus, said lithium salt A has two groups —OR each including at least one carbon-carbon double bond allowing the facilitated formation of a polymer containing a lithium salt.

According to a particular embodiment, said lithium salt A is of formula (III) Li—N(S(O)₂—OR)₂ with R of formula R⁴—X—(Y)ₙ— in which R⁴ is a radical comprising at least one carbon-carbon double bond; X is a heteroatom selected from the group consisting of O, S and N(R⁵) in which R⁵ is H or C₁-C₁₀ alkyl; preferably, R⁵ is H, CH₃ or C₂H₅; Y is a group selected from the group consisting of —C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹)C(R⁸)(R⁹)]— with R⁶ and R⁷ independently selected for each carbon atom and for each unit Y from H or F; R⁸ and R⁹ independently selected for each carbon atom and for each unit Y from H, F or CH₃; m an integer from 1 to 50, advantageously from 1 to 40, preferably from 1 to 30, more preferentially from 1 to 20, in particular from 1 to 10; z=0 if X is O; n is an integer from 1 to 50, advantageously from 1 to 40, preferably from 1 to 30, more preferentially from 1 to 20, in particular from 1 to 10.

When said lithium salt A is of formula (III) Li—N(S(O)₂—OR)₂, the substituents R are selected independently of each other for each of the functional groups S(O)₂—OR.

Thus, according to a preferred embodiment, said lithium salt A is of formula (IIIa), (IIIb), (IIIc), (IIId), (IIIe), (IIIf), (IIIg), (IIIh), (IIIi) or (IIIj):

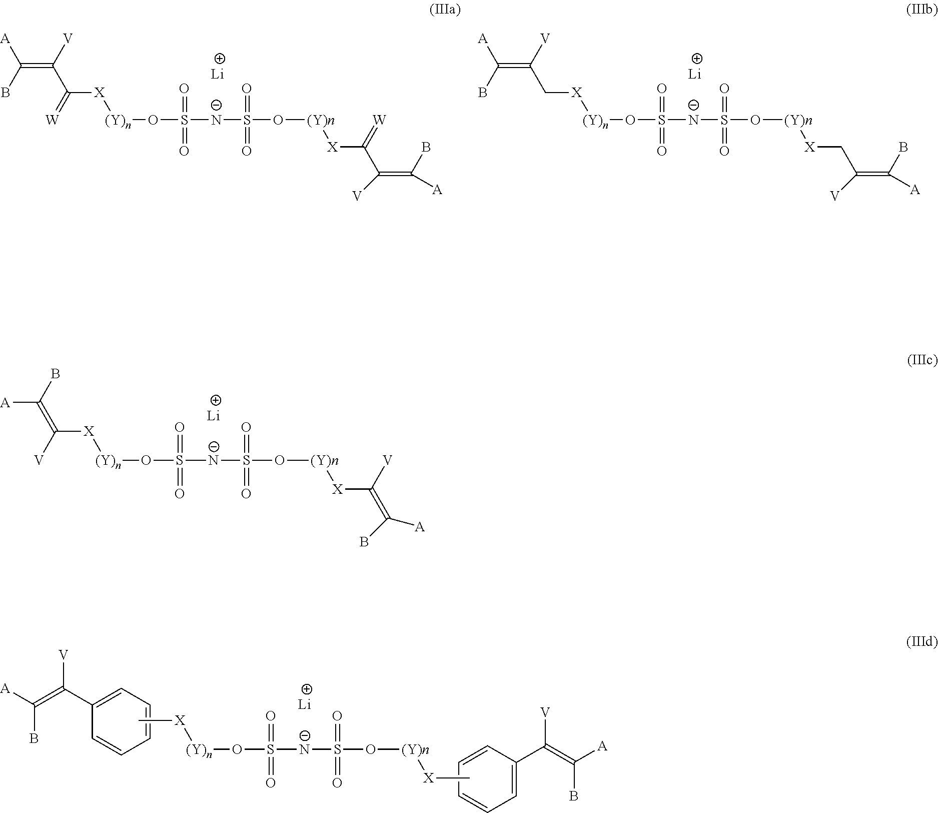

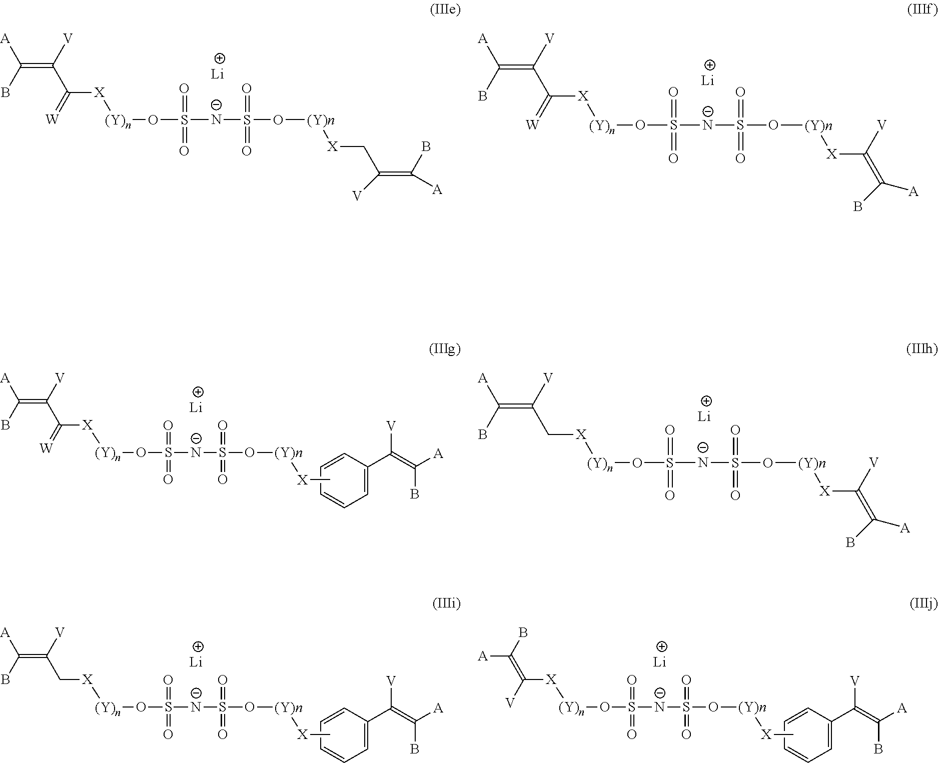

in which:
X is a heteroatom selected from the group consisting of O, S and N(R⁵) in which R⁵ is H or C₁-C₁₀ alkyl;
Y is a group selected from the group consisting of —C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹)C(R⁸)(R⁹)]— with R⁶ and R⁷ independently selected for each carbon atom and for each unit Y from H or F; R¹ and R⁹ independently selected for each carbon atom and for each unit Y from H, F or CH₃;
m is an integer ranging from 1 to 50, advantageously from 1 to 40, preferably from 1 to 30, more preferentially from 1 to 20 and in particular from 1 to 10;
z=0 if X is O;
n is an integer ranging from 1 to 50, advantageously from 1 to 40, preferably from 1 to 30, more preferentially from 1 to 20 and in particular from 1 to 10;
A, B and V are, independently of each other, selected from the group consisting of H, F, Cl, Br, I, C₁-C₂₀ alkyl optionally substituted with one or more substituents R″, C₂-C₂₀ alkenyl optionally substituted with one or more substituents R″, C₃-C₂₀ cycloalkyl optionally substituted with one or more substituents R″ or one or more C₁-C₅ alkyl groups, C₄-C₂₀ cycloalkenyl optionally substituted with one or more substituents R″ or one or more C₁-C₅ alkyl groups, C₆-C₂₀ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester; advantageously, A, B and V are, independently of each other, selected from the group consisting of H, F, Cl, Br, I, C₁-C₁₅ alkyl optionally substituted with one or more substituents R″, C₂-C₁₅ alkenyl optionally substituted with one or more substituents R″, C₃-C₁₅ cycloalkyl optionally substituted with one or more substituents R″ or one or more C₁-C₅ alkyl groups, C₄-C₁₅ cycloalkenyl optionally substituted with one or more substituents R″ or one or more C₁-C₅ alkyl groups, C₆-C₁₅ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably, F, C₁-C₄ ether, C₁-C₄ ester, C₁-C₄ thioether and C₁-C₄ thioester; preferably, A, B and V are, independently of each other, selected from the group consisting of H, F, Cl, Br, I, C₁-C₁₀ alkyl optionally substituted with one or more substituents R″, C₂-C₁₀ alkenyl optionally substituted with one or more substituents R″, C₃-C₁₀ cycloalkyl optionally substituted with one or more substituents R″ or one or more C₁-C₅ alkyl groups, C₄-C₁₀ cycloalkenyl optionally substituted with one or more substituents R″ or one or more C₁-C₅ alkyl groups, C₆-C₁₀ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester; more preferentially, A, B and V are, independently of each other, selected from the group consisting of H, F, Cl, Br, I, C₁-C₁₀ alkyl, C₂-C₁₀ alkenyl, C₃-C₁₀ cycloalkyl, C₄-C₁₀ cycloalkenyl, C₆-C₁₀ aryl, C₁-C₁₀ perfluoroalkyl, C₂-C₁₀ perfluoroalkenyl, C₃-C₁₀ perfluorocycloalkyl, C₄-C₁₀ perfluorocycloalkenyl and C₆-C₁₀ perfluoroaryl; in particular, A, B and V are, independently of each other, selected from the group consisting of H, F, Cl, Br, I, C₁-C₅ alkyl, C₁-C₅ perfluoroalkyl and C₆R¹⁰₅, with R¹⁰ independently selected from H, F and C₁-C₅ alkyl;
W is selected from O and S, preferably O.

Preferably, said lithium salt A is of formula (IIIa), (IIIb), (IIIc), (IIId), (IIIe), (IIIf), (IIIg), (IIIh), (IIIi) or (IIIj) in which
X is a heteroatom selected from the group consisting of O, S and N(R⁵) in which R⁵ is H or C₁-C₁₀ alkyl; preferably, R⁵ is H, CH₃ or C₂H₅;
Y is a group selected from the group consisting of —C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹)C(R⁸)(R⁹)]— with R⁶ and R⁷ independently selected for each carbon atom and for each unit Y from H or F; R⁸ and R⁹ independently selected for each carbon atom and for each unit Y from H, F or CH₃;
m is an integer ranging from 1 to 50, advantageously from 1 to 40, preferably from 1 to 30, more preferentially from 1 to 20 and in particular from 1 to 10;
z=0 if X is O;
n is an integer ranging from 1 to 50, advantageously from 1 to 40, preferably from 1 to 30, more preferentially from 1 to 20 and in particular from 1 to 10;
A and B are, independently of each other, selected from the group consisting of H, F, Cl, I, Br, CH₃, C₂H₅, CF₃, C₂F₅ and phenyl; preferably, A and B are identical and are selected from the group consisting of H, F, Cl, I and Br, or A is H and B is selected from the group consisting of F, Cl, Br, I, CH₃, C₂H₅, CF₃, C₂F₅ and phenyl;
V is selected from the group consisting of H, F, Cl, Br, I, C₁-C₅ alkyl, C₁-C₅ perfluoroalkyl and C₆R¹⁰₅ with R¹⁰ independently selected from H, F and C₁-C₅ alkyl;
W is selected from O and S, preferably O.

In particular, said lithium salt A is of formula (IIIa), (IIIb), (IIIc) or (IIId) as defined above.

Alternatively, said lithium salt A is of formula (IV) LiN(S(O)₂—OR)(S(O)₂R′) when said lithium salt B is of formula LiN(S(O)₂—F)(S(O)₂R′). Said lithium salt A is of formula (IV) LiN(S(O)₂—OR)(S(O)₂R′) in which R′ is a substituent selected from the group consisting of F, C₁-C₄ alkyl, C₁-C₄ perfluoroalkyl, C₁-C₄ alkoxy and C₁-C₄ perfluoroalkoxy and R is as defined above in the present patent application.

Thus, said lithium salt A is of formula (IV) LiN(S(O)₂—OR)(S(O)₂R′) in which
R′ is a substituent selected from the group consisting of F, C₁-C₄ alkyl, C₁-C₄ perfluoroalkyl, C₁-C₄ alkoxy and C₁-C₄ perfluoroalkoxy; preferably C₁-C₄ alkyl, C₁-C₄ perfluoroalkyl, C₁-C₄ alkoxy and C₁-C₄ perfluoroalkoxy;
R is of formula R⁴—X—(Y)ₙ— in which R⁴ is a radical comprising at least one carbon-carbon double bond; X is a heteroatom selected from the group consisting of O, S and N(R⁵) in which R⁵ is H or C₁-C₁₀ alkyl; preferably, R⁵ is H, CH₃ or C₂H₅; Y is a group selected from the group consisting of —C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹)C(R⁸)(R⁹)]— with R⁶ and R⁷ independently selected for each carbon atom and for each unit Y from H or F; R⁸ and R⁹ independently selected for each carbon atom and for each unit Y from H, F or CH₃; m an integer from 1 to 50, advantageously from 1 to 40, preferably from 1 to 30, more preferentially from 1 to 20, in particular from 1 to 10; z=0 if X is O; n is an integer from 1 to 50, advantageously from 1 to 40, preferably from 1 to 30, more preferentially from 1 to 20, in particular from 1 to 10.

Thus, said lithium salt A is of formula (IVa), (IVb), (IVc) or (IVd)

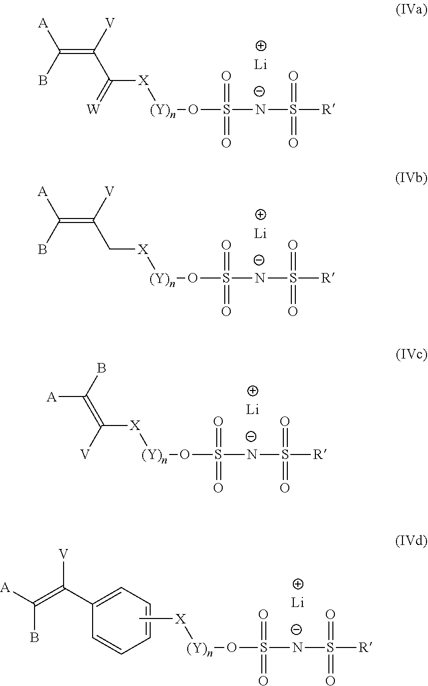

in which:
X is a heteroatom selected from the group consisting of O, S and N(R⁵) in which R⁵ is H or C₁-C₁₀ alkyl;
Y is a group selected from the group consisting of —C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹)C(R⁸)(R⁹)]— with R⁶ and R⁷ independently selected for each carbon atom and for each unit Y from H or F; R⁸ and R⁹ independently selected for each carbon atom and for each unit Y from H, F or CH₃;
m is an integer ranging from 1 to 50, advantageously from 1 to 40, preferably from 1 to 30, more preferentially from 1 to 20 and in particular from 1 to 10;
z=0 if X is O;
n is an integer ranging from 1 to 50, advantageously from 1 to 40, preferably from 1 to 30, more preferentially from 1 to 20 and in particular from 1 to 10;
A, B and V are, independently of each other, selected from the group consisting of H, F, Cl, Br, I, C₁-C₂₀ alkyl optionally substituted with one or more substituents R″, C₂-C₂₀ alkenyl optionally substituted with one or more substituents R″, C₃-C₂₀ cycloalkyl optionally substituted with one or more substituents R″ or one or more C₁-C₅ alkyl groups, C₄-C₂₀ cycloalkenyl optionally substituted with one or more substituents R″ or one or more C₁-C₅ alkyl groups, C₆-C₂₀ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester; advantageously, A, B and V are, independently of each other, selected from the group consisting of H, F, Cl, Br, I, C₁-C₁₅ alkyl optionally substituted with one or more substituents R″, C₂-C₁₅ alkenyl optionally substituted with one or more substituents R″, C₃-C₁₅ cycloalkyl optionally substituted with one or more substituents R″ or one or more C₁-C₅ alkyl groups, C₄-C₁₅ cycloalkenyl optionally substituted with one or more substituents R″ or one or more C₁-C₅ alkyl groups, C₆-C₁₅ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably, F, C₁-C₄ ether, C₁-C₄ ester, C₁-C₄ thioether and C₁-C₄ thioester; preferably, A, B and V are, independently of each other, selected from the group consisting of H, F, Cl, Br, I, C₁-C₁₀ alkyl optionally substituted with one or more substituents R″, C₂-C₁₀ alkenyl optionally substituted with one or more substituents R″, C₃-C₁₀ cycloalkyl optionally substituted with one or more substituents R″ or one or more C₁-C₅ alkyl groups, C₄-C₁₀ cycloalkenyl optionally substituted with one or more substituents R″ or one or more C₁-C₅ alkyl groups, C₆-C₁₀ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester; more preferentially, A, B and V are, independently of each other, selected from the group consisting of H, F, Cl, Br, I, C₁-C₁₀ alkyl, C₂-C₁₀ alkenyl, C₃-C₁₀ cycloalkyl, C₄-C₁₀ cycloalkenyl, C₆-C₁₀ aryl, C₁-C₁₀ perfluoroalkyl, C₂-C₁₀ perfluoroalkenyl, C₃-C₁₀ perfluorocycloalkyl, C₄-C₁₀ perfluorocycloalkenyl and C₆-C₁₀ perfluoroaryl; in particular, A, B and V are, independently of each other, selected from the group consisting of H, F, Cl, Br, I, C₁-C₅ alkyl, C₁-C₅ perfluoroalkyl and C₆R¹⁰₅, with R¹⁰ independently selected from H, F and C₁-C₅ alkyl;
W is selected from O and S, preferably O;
R′ is a substituent selected from the group consisting of F, C₁-C₄ alkyl, C₁-C₄ perfluoroalkyl, C₁-C₄ alkoxy and C₁-C₄ perfluoroalkoxy; preferably C₁-C₄ alkyl, C₁-C₄ perfluoroalkyl, C₁-C₄ alkoxy and C₁-C₄ perfluoroalkoxy.

Preferably, said lithium salt A is of formula (IVa), (IVb), (IVc) or (IVd) in which
X is a heteroatom selected from the group consisting of O, S and N(R⁵) in which R⁵ is H or C₁-C₁₀ alkyl; preferably, R⁵ is H, CH₃ or C₂H₅;
Y is a group selected from the group consisting of —C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹)C(R⁸)(R⁹)]— with R⁶ and R⁷ independently selected for each carbon atom and for each unit Y from H or F; R⁸ and R⁹ independently selected for each carbon atom and for each unit Y from H, F or CH₃;
m is an integer ranging from 1 to 50, advantageously from 1 to 40, preferably from 1 to 30, more preferentially from 1 to 20 and in particular from 1 to 10;
z=0 if X is O;
n is an integer ranging from 1 to 50, advantageously from 1 to 40, preferably from 1 to 30, more preferentially from 1 to 20 and in particular from 1 to 10;
A and B are, independently of each other, selected from the group consisting of H, F, Cl, I, Br, CH₃, C₂H₅, CF₃, C₂F₅ and phenyl; preferably, A and B are identical and are selected from the group consisting of H, F, Cl, I and Br, or A is H and B is selected from the group consisting of F, Cl, Br, I, CH₃, C₂H₅, CF₃, C₂F₅ and phenyl; V is selected from the group consisting of H, F, Cl, Br, I, C₁-C₅ alkyl, C₁-C₅ perfluoroalkyl and CR¹⁰₅ with R¹⁰ independently selected from H, F and C₁-C₅ alkyl;
W is selected from O and S, preferably O;
R′ is a substituent selected from the group consisting of F, C₁-C₄ alkyl, C₁-C₄ perfluoroalkyl, C₁-C₄ alkoxy and C₁-C₄ perfluoroalkoxy; preferably C₁-C₄ alkyl, C₁-C₄ perfluoroalkyl, C₁-C₄ alkoxy and C₁-C₄ perfluoroalkoxy.

### Catalyst D

Said process for preparing said lithium salt A is performed in the presence of a catalyst D. Advantageously, said catalyst D is a non-nucleophilic compound. The use of a non-nucleophilic compound helps to avoid degradation of said lithium salt B.

According to a preferred embodiment, said catalyst D is a compound with a pKa of greater than 11 measured at 25° C. in water. According to a particular embodiment, said catalyst D has a pKa of greater than 11.5, advantageously greater than 12.0, preferably greater than 12.5, in particular greater than 13.0.

Preferably, the mole ratio of the catalyst relative to said lithium salt B is greater than or equal to 0.1, preferably greater than or equal to 0.2. Preferably, the mole ratio of the catalyst to said lithium B salt is less than or equal to 2.5, preferably less than or equal to 2.

According to a preferred embodiment, said catalyst D is selected from the group consisting of N,N-diisopropylethylamine, 1,8-diazabicyclo(5.4.0)undec-7-ene, 6-(dibutylamino)-1,8-diazabicyclo[5.4.0]undec-7-ene, polystyrene-bound 1,8-diazabicyclo[5.4.0]undec-7-ene, 1,5,7-triazabicyclo[4.4.0]dec-5-ene, 7-methyl-1,5,7-triazabicyclo[4.4.0]dec-5-ene, 1,1,3,3-tetramethylguanidine, 2-tert-butyl-1,1,3,3-tetramethylguanidine, polystyrene-bound 1,5,7-triazabicyclo[4.4.0]dec-5-ene, 1,4-diazabicyclo[2.2.2]octane, quinuclidine, 1,5-diazabicyclo(4.3.0)non-5-ene, 2,6-di-tert-butylpyridine, 2,8,9-trimethyl-2,5,8,9-tetraaza-1-phosphabicyclo[3.3.3]undecane, cyclodiphosphazane, lithium diisopropylamide, sodium diisopropylamide, potassium diisopropylamide, magnesium diisopropylamide, calcium diisopropylamide, rubidium diisopropylamide, lithium bis(trimethylsilyl)amide, sodium bis(trimethylsilyl)amide, potassium bis(trimethylsilyl)amide, magnesium bis(trimethylsilyl)amide, calcium bis(trimethylsilyl)amide, rubidium bis(trimethylsilyl)amide, lithium tetramethylpiperidide, sodium tetramethylpiperidide, potassium tetramethylpiperidide, magnesium tetramethylpiperidide, calcium tetramethylpiperidide, rubidium tetramethylpiperidide, [18-crown-6]—KHF₂, KHF₂, N,N′-diisopropylimidazonium, bifluoride, tetrabutylammonium.

In particular, said catalyst D is selected from the group consisting of 1,8-diazabicyclo(5.4.0)undec-7-ene, 1,5,7-triazabicyclo[4.4.0]dec-5-ene, 2-tert-butyl-1,1,3,3-tetramethylguanidine, polystyrene-bound 1,5,7-triazabicyclo[4.4.0]dec-5-ene and polystyrene-bound 1,8-diazabicyclo[5.4.0]undec-7-ene.

### Implementation of the Process According to the Invention

The process may be performed under mild operating conditions. Step a) of the process is preferably performed in a reactor made of a material such as Inconel®, Hastelloy® or Monel®. According to a preferred embodiment, said process is performed at a temperature of from 10° C. to 70° C., advantageously from 10° C. to 50° C., preferably at a temperature of from 10° C. to 30° C.

Said process may be performed at a pressure of from 0.5 to 3 bara, preferably at atmospheric pressure, although the pressure is not an important parameter in the implementation of the process.

Said process may be performed under an atmosphere that may or may not be inert.

Preferably, in step a), said lithium salt B is placed in contact with said catalyst D followed by the addition of said compound C. This enables improved conversion of the reaction between the lithium salt and compound C.

The reaction between the lithium salt B and compound C is preferably performed at stoichiometry or with a slight excess of or deficient in compound C. Thus, for example, if said lithium salt B includes two S(O)₂—F groups, 2 eq. of compound C relative to the number of moles of lithium salt B are added.

Said process may be performed in the presence of a solvent, although the process may be performed without a solvent. When a solvent is used, this may be, for example, acetonitrile, benzonitrile, dimethyl sulfoxide, dimethylformamide, 1,4-dioxane, nitromethane, dimethyl carbonate, diethyl carbonate, propyl carbonate, ethylene carbonate, N-methyl-2-pyrrolidone or a mixture thereof.

In addition to the lithium salt A, said product mixture may comprise said lithium salt B which has not reacted, or said compound C which has not reacted, or a mixture of the two.

Said product mixture may also comprise F—SiR¹R²R³ with R¹, R² and R³ as defined in the present patent application. When R¹, R² and R³ are CH₃, the F—Si(CH₃)₃ formed in step a) is readily removed given its boiling point (Bp=16° C.).

When said lithium salt B has two S(O)₂—F groups, said product mixture may also comprise a compound in which only one of the fluorine atoms has been substituted. For example, when said lithium salt B is LiN(S(O)₂—F)₂, said product mixture may comprise LiN(S(O)₂F)(S(O)₂—OR).

Said lithium salt B may optionally contain impurities related to its preparation process, such as LiCl, LiF or FSO₃Li in a mass content of less than 1000 ppm, advantageously less than 500 ppm, preferably less than 100 ppm, relative to the total weight of said lithium salt. In particular, FSO₃Li, if present, represents a mass content of less than 5 ppm relative to the total weight of said lithium salt B. Other impurities may also be present in said lithium salt B as described in WO 2018/104674. These impurities may optionally be present in said lithium A salt in a content less than or equal to that mentioned for said lithium salt B.

According to a preferred embodiment, said process also comprises a step b) of purifying said mixture of products obtained in step a) to isolate said lithium salt A. If step a) has been performed in the presence of a solvent, the latter is preferably evaporated off prior to performing step b). Said step b) notably comprises a step of extracting said lithium A salt with an apolar organic solvent. Said apolar organic solvent is, for example, a C₅-C₁₀ alkyl, C₅-C₁₀ cycloalkyl, C₆-C₁₀ aryl, C₁-C₃ haloalkyl, diethyl ether, 1,4-dioxane, tetrahydrofuran or a mixture thereof. Preferably, said apolar organic solvent is n-hexane, pentane, heptane, cyclohexane, cyclopentane, benzene, toluene, xylene, cumene, CCl₄, CHCl₃, diethyl ether, 1,4-dioxane, tetrahydrofuran or a mixture thereof.

Said process according to the present invention affords said lithium salt A in very high purity. The mixture obtained in step b) and comprising said lithium salt and said apolar solvent is distilled to remove the apolar solvent and recover said lithium salt A. The implementation of step b) results in a composition comprising at least 95% by weight, preferably at least 98% by weight, in particular at least 99% by weight, more particularly at least 99.5% of lithium salt A relative to the total weight of the composition.

Said composition may comprise less than 1% and preferably less than 0.5% by weight of additional compounds selected from the group consisting of said compound C, said compound B and F—SiR¹R²R³ with R¹, R² and R³ as defined in the present patent application. When said lithium salt B is LiN(S(O)₂—F)₂, said composition may comprise less than 1% and preferably less than 0.5% by weight of additional compounds selected from the group consisting of said compound C, said compound B, F—SiR¹R²R³ and LiN(S(O)₂F)(S(O)₂—OR) with R, R¹, R² and R³ as defined in the present application.

### Lithium Salt of Formula (IIIa), (IIIb), (IIIc), (IIId), (IIIe), (IIIf), (IIIg), (IIIh), (IIIi), (IIIj), (IVa), (IVb), (IVc) or (IVd).

According to a second aspect of the present invention, a lithium salt is provided. According to a preferred embodiment, said lithium salt is of formula (IIIa), (IIIb), (IIIc), (IIId), (IIIe), (IIIf), (IIIg), (IIIh), (IIIi), (IIIi), (IVa), (IVb), (IVc) or (IVd)

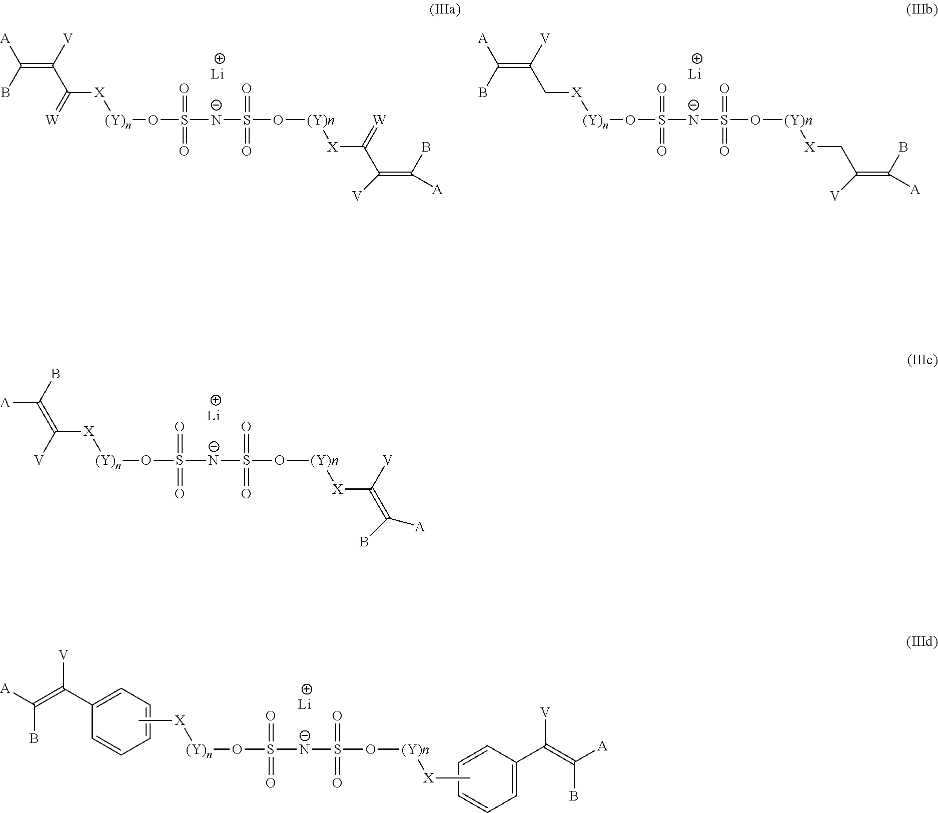

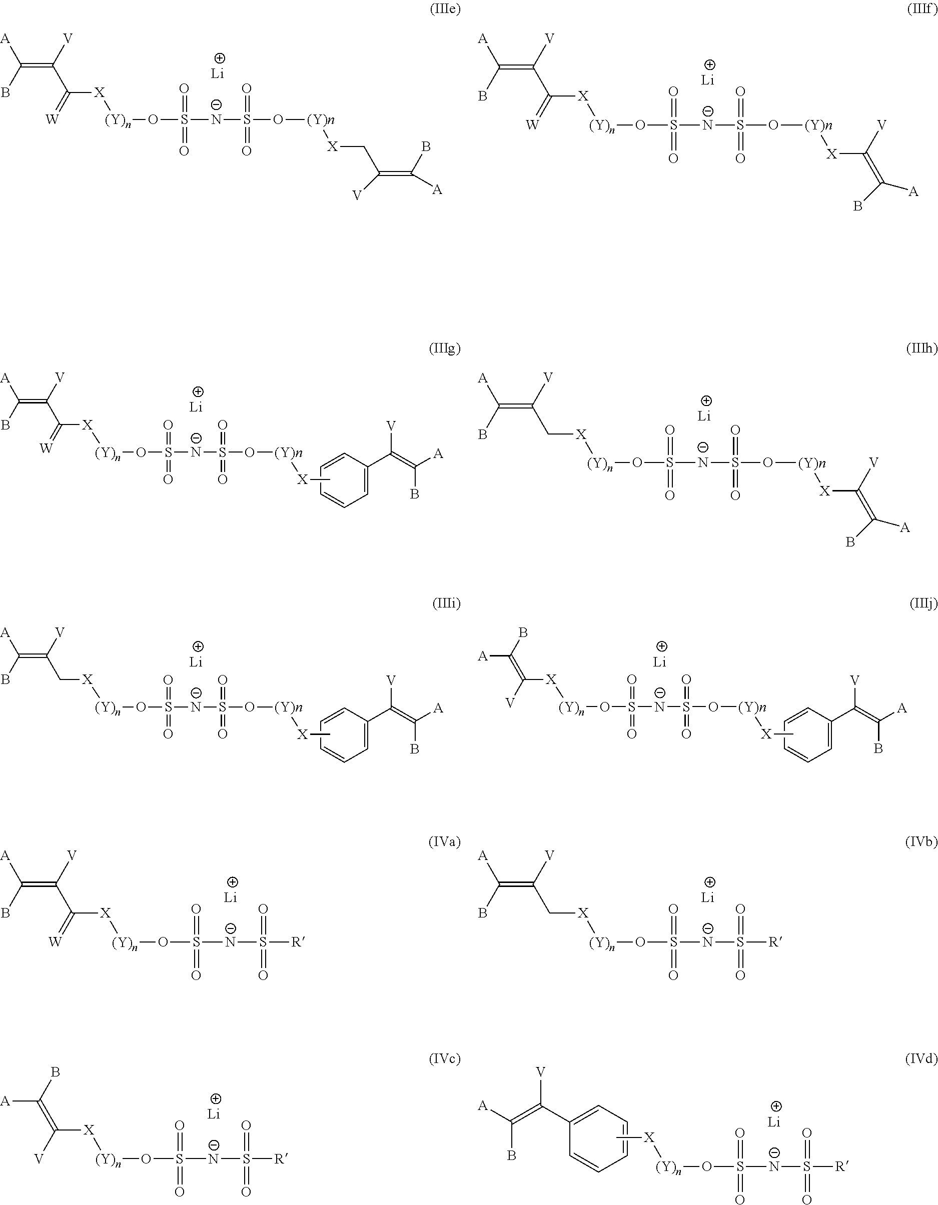

in which:
X is a heteroatom selected from the group consisting of O, S and N(R⁵) in which R⁵ is H or C₁-C₁₀ alkyl;
Y is a group selected from the group consisting of —C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹)C(R⁸)(R⁹)]— with R⁶ and R⁷ independently selected for each carbon atom and for each unit Y from H or F; R¹ and R⁹ independently selected for each carbon atom and for each unit Y from H, F or CH₃;
m is an integer ranging from 1 to 50, advantageously from 1 to 40, preferably from 1 to 30, more preferentially from 1 to 20 and in particular from 1 to 10;
z=0 if X is O;
n is an integer ranging from 1 to 50, advantageously from 1 to 40, preferably from 1 to 30, more preferentially from 1 to 20 and in particular from 1 to 10;
A, B and V are, independently of each other, selected from the group consisting of H, F, Cl, Br, I, C₁-C₂₀ alkyl optionally substituted with one or more substituents R″, C₂-C₂₀ alkenyl optionally substituted with one or more substituents R″, C₃-C₂₀ cycloalkyl optionally substituted with one or more substituents R″ or one or more C₁-C₅ alkyl groups, C₄-C₂₀ cycloalkenyl optionally substituted with one or more substituents R″ or one or more C₁-C₅ alkyl groups, C₆-C₂₀ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester; advantageously, A, B and V are, independently of each other, selected from the group consisting of H, F, Cl, Br, I, C₁-C₁₅ alkyl optionally substituted with one or more substituents R″, C₂-C₁₅ alkenyl optionally substituted with one or more substituents R″, C₃-C₁₅ cycloalkyl optionally substituted with one or more substituents R″ or one or more C₁-C₅ alkyl groups, C₄-C₁₅ cycloalkenyl optionally substituted with one or more substituents R″ or one or more C₁-C₅ alkyl groups, C₆-C₁₅ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably, F, C₁-C₄ ether, C₁-C₄ ester, C₁-C₄ thioether and C₁-C₄ thioester; preferably, A, B and V are, independently of each other, selected from the group consisting of H, F, Cl, Br, I, C₁-C₁₀ alkyl optionally substituted with one or more substituents R″, C₂-C₁₀ alkenyl optionally substituted with one or more substituents R″, C₃-C₁₀ cycloalkyl optionally substituted with one or more substituents R″ or one or more C₁-C₅ alkyl groups, C₄-C₁₀ cycloalkenyl optionally substituted with one or more substituents R″ or one or more C₁-C₅ alkyl groups, C₆-C₁₀ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester; more preferentially, A, B and V are, independently of each other, selected from the group consisting of H, F, Cl, Br, I, C₁-C₁₀ alkyl, C₂-C₁₀ alkenyl, C₃-C₁₀ cycloalkyl, C₄-C₁₀ cycloalkenyl, C₆-C₁₀ aryl, C₁-C₁₀ perfluoroalkyl, C₂-C₁₀ perfluoroalkenyl, C₃-C₁₀ perfluorocycloalkyl, C₄-C₁₀ perfluorocycloalkenyl and C₆-C₁₀ perfluoroaryl; in particular, A, B and V are, independently of each other, selected from the group consisting of H, F, Cl, Br, I, C₁-C₅ alkyl, C₁-C₅ perfluoroalkyl and C₆R¹⁰₅, with R¹⁰ independently selected from H, F and C₁-C₅ alkyl;
W is selected from O and S, preferably O;
R′ is a substituent selected from the group consisting of F, C₁-C₄ alkyl, C₁-C₄ perfluoroalkyl, C₁-C₄ alkoxy and C₁-C₄ perfluoroalkoxy; preferably C₁-C₄ alkyl, C₁-C₄ perfluoroalkyl, C₁-C₄ alkoxy and C₁-C₄ perfluoroalkoxy.

Preferably, said lithium salt is of formula (IIIa), (IIIb), (IIIc), (IIId), (IIIe), (IIIf), (IIIg), (IIIh), (IIIi), (IIIj), (IVa), (IVb), (IVc) or (IVd) in which
X is a heteroatom selected from the group consisting of O, S and N(R⁵) in which R⁵ is H or C₁-C₁₀ alkyl; preferably, R⁵ is H, CH₃ or C₂H₅;
Y is a group selected from the group consisting of —C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹)C(R⁸)(R⁹)]— with R⁶ and R⁷ independently selected for each carbon atom and for each unit Y from H or F; R⁸ and R⁹ independently selected for each carbon atom and for each unit Y from H, F or CH₃;
m is an integer ranging from 1 to 50, advantageously from 1 to 40, preferably from 1 to 30, more preferentially from 1 to 20 and in particular from 1 to 10;
z=0 if X is O;
n is an integer ranging from 1 to 50, advantageously from 1 to 40, preferably from 1 to 30, more preferentially from 1 to 20 and in particular from 1 to 10;
A and B are, independently of each other, selected from the group consisting of H, F, Cl, I, Br, CH₃, C₂H₅, CF₃, C₂F₅ and phenyl; preferably, A and B are identical and are selected from the group consisting of H, F, Cl, I and Br, or A is H and B is selected from the group consisting of F, Cl, Br, I, CH₃, C₂H₅, CF₃, C₂F₅ and phenyl;
V is selected from the group consisting of H, F, Cl, Br, I, C₁-C₅ alkyl, C₁-C₅ perfluoroalkyl and CR¹⁰₅ with R¹⁰ independently selected from H, F and C₁-C₅ alkyl;
W is selected from O and S, preferably O;
R′ is a substituent selected from the group consisting of F, C₁-C₄ alkyl, C₁-C₄ perfluoroalkyl, C₁-C₄ alkoxy and C₁-C₄ perfluoroalkoxy; preferably C₁-C₄ alkyl, C₁-C₄ perfluoroalkyl, C₁-C₄ alkoxy and C₁-C₄ perfluoroalkoxy.

In particular, said lithium salt is of formula (IIIa), (IIIb), (IIIc), (IIId), (IIIe), (IIIf), (IIIg), (IIIh), (IIIi), (IIIj), (IVa), (IVb), (IVc) or (IVd) in which
A and B are, independently of each other, selected from the group consisting of H, F, Cl, I, Br, CH₃, C₂H₅, CF₃, C₂F₅ and phenyl; preferably, A and B are identical and are selected from the group consisting of H, F, Cl, I and Br, or A is H and B is selected from the group consisting of F, Cl, Br, I, CH₃, C₂H₅, CF₃, C₂F₅ and phenyl;
V is selected from the group consisting of H, F, Cl, Br, I, C₁-C₅ alkyl, C₁-C₅ perfluoroalkyl and CR¹⁰₅ with R¹⁰ independently selected from H, F and C₁-C₅ alkyl;
W is O;
X is O;
Y is a group selected from the group consisting of CH₂CH₂, CH₂CF₂, CF₂CF₂, CH₂CHF, CF₂CH₂ and CHFCH₂, and n is an integer from 1 to 25; or
Y is a group —[(C(R⁸)(R⁹)—C(R⁸)(R⁹)O)ₘ—C(R⁸)(R⁹)C(R⁸)(R⁹)]— with R⁸ and R⁹ independently selected for each carbon atom and for each unit Y from H, F or CH₃, m is an integer from 1 to 25 and n is 1;
R′ is a substituent selected from the group consisting of F, C₁-C₄ alkyl, C₁-C₄ perfluoroalkyl, C₁-C₄ alkoxy and C₁-C₄ perfluoroalkoxy; preferably C₁-C₄ alkyl, C₁-C₄ perfluoroalkyl, C₁-C₄ alkoxy and C₁-C₄ perfluoroalkoxy.

According to a preferred embodiment, said lithium salt is of formula (IIIa) in which:
A and B are H,
V is CH₃ or H,
W is O,
X is O and
Y is a group selected from the group consisting of CH₂CH₂, CH₂CF₂, CF₂CF₂, CH₂CHF, CF₂CH₂ and CHFCH₂, and n is an integer from 1 to 10; or
Y is a group —[(C(R⁸)(R⁹)—C(R⁸)(R⁹)O)ₘ—C(R⁸)(R⁹)C(R⁸)(R⁹)]— with R⁸ and R⁹ independently selected for each carbon atom and for each unit Y from H, F or CH₃, m is an integer from 1 to 25 and n is 1.

According to another aspect of the present invention, a polymer is provided. Said polymer comprises monomer units derived from at least one of the lithium salts according to the present invention, i.e. according to any one of the formulae (IIIa), (IIIb), (IIIc), (IIId), (IIIe), (IIIf), (IIIg), (IIIh), (IIIi), (IIIj), (IVa), (IVb), (IVc) or (IVd). Said polymer may comprise monomer units derived from one of the lithium salts according to the present invention, or monomer units derived from several of these salts. Said lithium salts can polymerize via their vinyl function.

According to a preferred embodiment, said polymer also comprises monomer units resulting from a monomer M1 of formula R¹R²C═C(R³)C(O)R in which the substituents R¹, R² and R³ are selected, independently of each other, from the group consisting of H and C₁-C₅ alkyl; R is selected from the group consisting of —NHC(CH₃)₂CH₂C(O)CH₃ or —OR′ with R′ selected from the group consisting of H and C₁-C₁₈ alkyl optionally substituted with one or more —OH groups or a 5- or 10-membered heterocycle comprising at least one nitrogen atom in its cyclic chain. Said heterocycle may be saturated or unsaturated or aromatic. Said heterocycle may be monocyclic or bicyclic. Said heterocycle may be a pyrrole, pyrrolidine, pyridine, piperidine, pyrimidine, pyrazine, 1,4-dihydropyridine, indole, oxindole, isatin, quinoline, isoquinoline, quinazoline, imidazoline, pyrazolidine, 2-pyrrolidone, delta-lactam, succinimide, 2-imidazolidinone or 4-imidazolidinone ring. Said heterocycle may be substituted with one or more C₁-C₅ alkyl groups. As mentioned above, the C₁-C₁₈ alkyl is optionally substituted with said heterocycle. The latter may be bonded to the chain via the nitrogen atom or any other atoms forming the heterocycle. Preferably, the heterocycle is 2-pyrrolidone, delta-lactam, succinimide, 2-imidazolidinone or 4-imidazolidinone.

Preferably, said monomer M1 has the formula R¹R²C═C(R³)C(O)R in which the substituents R¹, R² and R³ are, independently of each other, selected from the group consisting of H and C₁-C₅ alkyl; R is —OR′ with R′ selected from the group consisting of H and C₁-C₁₈ alkyl optionally substituted with one or more —OH groups. According to a preferred embodiment, the substituent R′ is selected from the group consisting of H, methyl, ethyl, propyl, n-butyl, isobutyl, t-butyl, n-dodecyl, amyl, isoamyl, hexyl, 2-ethylhexyl, lauryl, n-octyl, hydroxybutyl, hydroxypropyl and ethyl, substituted with a ureido, hydroxyethyl, hydroxypropyl or hydroxybutyl group.

In particular, said monomer M1 is of formula R¹R²C═C(R³)C(O)R in which the substituents R¹ and R² are H; R³ is H or CH₃; R is —OR′ with R′ selected from the group consisting of H, methyl, ethyl, propyl, n-butyl, isobutyl, t-butyl, hydroxypropyl, hydroxybutyl, 2-pyrrolidone, deltalactam, succinimide, 2-imidazolidinone and 4-imidazolidinone.

Thus, said monomer M1 may be acrylic acid, methyl acrylate, ethyl acrylate, propyl acrylate, n-butyl acrylate, isobutyl acrylate, t-butyl acrylate, n-dodecyl acrylate, amyl acrylate, isoamyl acrylate, hexyl acrylate, 2-ethylhexyl acrylate, diacetone acrylamide, lauryl acrylate, n-octyl acrylate, hydroxypropyl methacrylate, hydroxybutyl acrylate, methyl acrylic acid, methyl methacrylate, ethyl methacrylate, propyl methacrylate, n-butyl methacrylate, isobutyl methacrylate, t-butyl methacrylate, n-dodecyl methacrylate, amyl methacrylate, isoamyl methacrylate, hexyl methacrylate, 2-ethylhexyl methacrylate, lauryl methacrylate, n-octyl methacrylate, ureido methacrylate and the mixtures thereof. Among these, the alkyl acrylates with an alkyl group containing from 1 to 8 carbon atoms are preferred, and the alkyl acrylates with an alkyl group containing from 1 to 5 carbon atoms are more preferable. These compounds can be used alone or as a mixture of two or more.

According to a preferred embodiment, said polymer also comprises monomer units resulting from a monomer M2 selected from the group consisting of vinylidene fluoride, vinyl fluoride, trifluoroethylene, chlorotrifluoroethylene, 1,2-difluoroethylene, tetrafluoroethylene, hexafluoropropylene, perfluoro(alkyl vinyl) ethers, perfluoro(1,3-dioxole), perfluoro(2,2-dimethyl-1,3-dioxole), the monomer of formula CF₂═CFOCF₂CF(CF₃)OCF₂CF₂X in which X is SO₂F, CO₂H, CH₂OH, CH₂OCN or CH₂OPO₃H, the monomer of formula CF₂═CFOCF₂CF₂SO₂F, the monomer of formula F(CF₂)nCH₂OCF═CF₂ in which n is 1, 2, 3, 4 or 5, the monomer of formula R¹CH₂OCF═CF₂ in which R′ is hydrogen or F(CF₂)m and m has the value 1, 2, 3 or 4, the monomer of formula R²OCF═CH₂ in which R² is F(CF₂)p and p is 1, 2, 3 or 4, perfluorobutylethylene, trifluoropropene, tetrafluoropropene, hexafluoroisobutylene, perfluorobutylethylene, pentafluoropropene, bromotrifluoroethylene, chlorofluoroethylene, chlorotrifluoropropene or 2-trifluoromethyl-3,3,3-trifluoro-1-propene. Preferably, said monomer M2 is selected from the group consisting of vinylidene fluoride, trifluoroethylene, chlorotrifluoroethylene, tetrafluoroethylene, hexafluoropropylene, perfluoro(alkyl vinyl) ethers, perfluoro(1,3-dioxole), perfluoro(2,2-dimethyl-1,3-dioxole), the monomer of formula CF₂═CFOCF₂CF(CF₃)OCF₂CF₂X in which X is SO₂F, CO₂H, CH₂OH, CH₂OCN or CH₂OPO₃H, the monomer of formula CF₂═CFOCF₂CF₂SO₂F, the monomer of formula F(CF₂)nCH₂OCF═CF₂ in which n is 1, 2, 3, 4 or 5, the monomer of formula R¹CH₂OCF═CF₂ in which R¹ is hydrogen or F(CF₂)m and m has the value 1, 2, 3 or 4, the monomer of formula R²OCF═CH₂ in which R² is F(CF₂)ₚ and p is 1, 2, 3 or 4, tetrafluoropropene or chlorotrifluoropropene. In particular, said monomer M2 is selected from the group consisting of vinylidene fluoride, trifluoroethylene, chlorotrifluoroethylene, tetrafluoroethylene, hexafluoropropylene, tetrafluoropropene and chlorotrifluoropropene. Said polymer may also comprise monomer units derived from a monomer M3 selected from the group consisting of fumaric acid, crotonic acid, itaconic acid and the like; vinyl ester compounds such as vinyl acetate, vinyl neodecanoate and the like; amide compounds such as acrylamide, methacrylamide, N-methylacrylamide, N-methylmethacrylamide, N-methylolacrylamide, N-methylolmethacrylamide, N-alkylacrylamide, N-alkylmethacrylamide, N,N-dialkylacrylamide, N,N-dialkylmethacrylamide, diacetone acrylamide and the like; N-dialkylaminoethyl acrylate, glycidyl acrylate, n-dodecyl acrylate, fluoroalkyl acrylate and the like; dialkylaminoethyl methacrylate, fluoroalkyl methacrylate, glycidyl methacrylate, ethylene glycol dimethacrylate and the like; maleic anhydride, and alkenyl glycidyl ether compounds such as allyl glycidyl ether and the like; conjugated dienes such as 1,3-butadiene, isoprene and the like; divinyl hydrocarbon-based compounds such as divinylbenzene and the like; and alkenyl cyanides such as acrylonitrile, methacrylonitrile and the like. These compounds may be used alone or as a mixture of two or more. Among these, the preferred compounds are 1,3-butadiene and acrylonitrile.

The polymer as described in the present patent application may be used in many applications. Thus, said polymer may be used as a binder for an electrode (cathode or anode) or as a coating for a separator.

Thus, according to another aspect, the present invention provides an electrode composition comprising said polymer according to the present invention, an active material and optionally a conductive agent.

In a preferred embodiment, the electrode composition has the following mass composition:
a. 50% to 99.9% of active material, preferably 50% to 99%,
b. 25% to 0% of conductive agent, preferably 25% to 0.5%,
c. 25% to 0.05% of said binder according to the invention, preferably 25% to 0.5%,
d. 0% to 5% of at least one additive chosen from the group consisting of a plasticizer, an ionic liquid, a dispersing agent for conductive additive, and a flow aid agent,
the sum of all these percentages being 100%.

The conductive agents in the electrode are composed of one or more materials which can improve the conductivity. Some examples comprise carbon blacks, such as acetylene black or Ketjen black: carbon fibers, such as a carbon nanotube, a carbon nanofiber or a vapor grown carbon fiber; or metal powders, such as an SUS powder and an aluminum powder.

The active materials in the electrode compositions are materials which are capable of storing and releasing lithium ions.

In a preferred embodiment, said electrode is a negative electrode. In particular, for a negative electrode, said active material is chosen from the group consisting of a lithium alloy, lithium metal, a metal oxide, a carbon material, such as graphite or hard carbon, silicon, silicone, an alloy of silicon and Li₄TiO₁₂. The form of the negative electrode active material is not particularly limited but is preferably particulate.

In another preferred embodiment, said electrode is a positive electrode. Preferably, for a positive electrode, said active material is chosen from the group consisting of LiCoO₂, Li(Ni, Co, AI)O₂, Li₍₁₊ₓ₎NiₐMn_{b}Co_{c} (x representing a real number of 0 or more, a=0.8, 0.6, 0.5 or ⅓, b=0.1, 0.2, 0.3 or ⅓, c=0.1, 0.2 or ⅓), LiNiO₂, LiMn₂O₄, LiCoMnO₄, Li₃NiMn₃O₃, Li₃Fe₂(PO₄)₃, Li₃V₂(PO₄)₃, an Li Mn spinel substituted with a different element having a composition represented by Li₁₊ₓMn_{2-x-y}M_{y}O₄, M representing at least one metal chosen from Al, Mg, Co, Fe, Ni and Zn, x and y independently representing a real number of between 0 and 2, lithium titanate LiₓTiO_{y}, x and y independently representing a real number of between 0 and 2, and a lithium metal phosphate having a composition represented by LiMPO₄, M representing Fe, Mn, Co or Ni. The form of the positive electrode active material is not particularly limited but is preferably particulate. Furthermore, the surface of each of the materials described above can be coated. The coating material is not particularly limited as long as it has a conductivity for lithium ions and contains a material capable of being maintained in the form of a coating layer on the surface of the active material. Examples of the coating material comprise LiNbO₃, Li₄Ti₅O₁₂ and Li₃PO₄. Said electrode composition can be deposited on at least one face of a current collector in order to form said electrode. This deposition may be performed in the presence of an organic solvent, water, a mixture of the two or via a solvent-free process. Said organic solvent can be selected from the group consisting of N-methylpyrrolidone (NMP), dimethyl sulfoxide (DMSO), N,N-dimethylformamide (DMF), triethyl phosphite (TEP), acetone, cyclopentanone, tetrahydrofuran, methyl ethyl ketone (MEK), methyl isobutyl ketone (MiBK), ethyl acetate (EA), butyl acetate (BA), ethylene carbonate (EC), propylene carbonate (PC), dimethyl carbonate (DMC), diethyl carbonate (DEC), ethyl methyl carbonate (EMC), γ-butyrolactone and N-butylpyrrolidone; and mixtures thereof.

According to another aspect of the present invention, an Li-ion battery is provided. Preferably, the Li-ion battery comprises a positive electrode, a negative electrode and a separator, at least one electrode being an electrode according to the present invention.

According to another aspect of the present invention, said polymer according to the present invention may be used as coating in a separator arranged between two electrodes. Said separator according to the present invention comprises a coating comprising, preferably consisting of, the polymer according to the present invention, optionally arranged on one or both faces of a porous support. In this case, the coating is used to coat the support of a separator, on at least one face, in the form of a monolayer or of multilayers. There is no specific limitation on the choice of the support which is coated with the coating of the invention, as long as it is a porous substrate having pores. When it comprises several layers, the coating as described in the present invention is arranged on the external face of the support, that is to say on the face which will be first in contact with the electrolyte composition used in the battery. Advantageously, the application of the coating to the support takes place by the aqueous route or by the solvent route. The porous substrate may be in the form of a membrane or a fibrous fabric. When the porous substrate is fibrous, it may be a nonwoven web forming a porous web, such as a web obtained by direct spinning or melt blowing (of spun bond or melt blown type) or electrospinning. Examples of porous substrates that are useful in the invention as support comprise, without being limited thereto: polyolefins, polyethylene terephthalate, polybutylene terephthalate, polyester, polyacetal, polyamide, polycarbonate, polyimide, polyether ether ketone, polyether sulfone, poly(phenylene oxide), poly(phenylene sulfide), polyethylene naphthalate or mixtures thereof. However, other engineering plastics which are heat-resistant may be used, without specific limitation. Nonwoven materials made of natural or synthetic materials may also be used as substrate of the separator. The porous substrate generally has a thickness of from 1 to 50 μm, and typically consists of membranes obtained by extrusion and drawing (wet or dry process) or cast nonwovens. The porous substrate preferably has a porosity of between 5% and 95%. The average size of the pores (diameter) is preferably of between 0.001 and 50 μm, more preferably between 0.01 and 10 μm. The support may also be aluminum or aluminum coated with a polymer layer.

In addition to said composition, the coating for a separator may contain inorganic particles which serve to form micropores in the coating (the interstices between inorganic particles). The addition of inorganic particles can also contribute to the heat resistance or improve the wettability. According to one embodiment, said coating comprises from 50% to 99% by weight of inorganic particles, relative to the weight of the coating. These inorganic particles must be electrochemically stable (not subject to oxidation and/or to reduction within the range of voltages which are used). In addition, the pulverulent inorganic materials preferably have a high ionic conductivity. Low-density materials are preferred to materials of higher density, since the weight of the battery produced can be reduced. The dielectric constant is preferably equal to or greater than 5. According to one embodiment, said inorganic particles are chosen from the group consisting of: BaTiO₃, Pb(Zr,Ti)O₃, Pb₁₋ₓLaₓZr_{y}O₃ (0<x<1, 0<y<1), PBMg₃Nb_{2/3})₃, PbTiO₃, hafnia (HfO (HfO₂)), SrTiO₃, SnO₂, CeO₂, MgO, NiO, CaO, ZnO, Y₂O₃, boehmite (γ-AIO(OH)), Al₂O₃, TiO₂, SiC, ZrO₂, boron silicate, BaSO₄, nanoclays or mixtures thereof. The coating for a separator can optionally comprise from 0% to 15% by weight, relative to the polymer, and preferably from 0.1% to 10% by weight of additives chosen from thickeners, pH-adjusting agents, anti-settling agents, surfactants, wetting agents, fillers, anti-foaming agents and fugitive or non-fugitive adhesion promoters. The fillers mentioned here in the additives are different from the inorganic particles mentioned above.

According to another aspect of the present invention, an Li-ion battery is provided. Preferably, the Li-ion battery comprises a positive electrode, a negative electrode and said separator according to the present invention.

### EXAMPLES

### Example 1

In a 50-ml reactor, 1.06 g LiFSI are dissolved in 20 ml acetonitrile at room temperature (22° C.). 1.5 ml of 1,8-diazabicyclo(5.4.0)undec-7-ene (2 eq. relative to the LiFSI—pKa of 13.5 at 25° C. in water) are added to this solution and the resulting mixture is stirred for 5 minutes, followed by addition of 2.54 ml of 2-(trimethylsiloxy)ethyl methacrylate (2 eq. relative to the LiFSI). The reaction mixture is stirred at room temperature for 24 hours under the ambient atmosphere. The acetonitrile is evaporated off using a rotary evaporator at 30° C. and 1 mbar. The residue is mixed with petroleum ether to produce two phases. The upper phase containing lithium salt A and petroleum ether is separated out, and the petroleum ether is evaporated off using a rotary evaporator at 30° C. and 1 mbar. The product recovered is the lithium salt of formula (IIIa) with A and B═H, V═CH₃, W═O, X═O and Y is CH₂CH₂; n=1. The conversion of the LiFSI is 100%. The purity of the lithium salt is greater than 99%.

### Example 2

Example 1 is reproduced with 0.17 ml of 1,8-diazabicyclo(5.4.0)undec-7-ene (0.2 eq. relative to the LiFSI). The product recovered is the lithium salt of formula (IIIa) with A and B═H, V═CH₃, W═O, X═O and Y is CH₂CH₂; n=1. The conversion of the LiFSI is 100%. The purity of the lithium salt is greater than 99%.

### Example 3

Example 1 is reproduced with 0.17 ml of 1,8-diazabicyclo(5.4.0)undec-7-ene (0.2 eq. relative to the LiFSI), and 2 molar equivalents of p-(trimethylsilyloxy)styrene. The product recovered is the lithium salt of formula (IIId) with A and B═H, V═H, X═O. The conversion of the LiFSI is 79%. The purity of the lithium salt is greater than 99%.

### Example 4

Example 1 is reproduced with 0.17 ml of 1,8-diazabicyclo(5.4.0)undec-7-ene (0.2 eq. relative to the LiFSI), 1 molar equivalent of p-(trimethylsilyloxy)styrene and 1 molar equivalent of 2-(trimethoxysilyl)ethyl methacrylate. The product recovered is the lithium salt of formula (IIIg) with A and B═H, V═H, X═O, and A and B═H, V═CH₃, W═O, X═O and Y is CH₂CH₂; n=1. The conversion of the LiFSI is 83%.

### Comparative Example 1

Example 1 is reproduced with 2.16 ml triethylamine (2 eq. relative to the LiFSI). Triethylamine has a pKa of less than 11. The product recovered is the lithium salt of formula (IIIa) with A and B═H, V═CH₃, W═O, X═O and Y is CH₂CH₂; n=1. The conversion of the LiFSI is 8%.

### Example 5

To polymerize the monomers according to the present invention, the following procedure is followed. 3 g of water are introduced into a reactor equipped with a mixing and heating device. The temperature is set at 95° C. Over a period of 2 hours, 3 g of a mixture of the compound of formula (IIIa) and methacrylic acid (10/90 molar percentage) and 0.4 g of sodium persulfate are introduced into 7 g of water. The temperature is maintained for 2 hours. The reactor is cooled to room temperature.

1. A process for preparing a lithium salt A, comprising a step a) of placing in contact a lithium salt B comprising at least one —S(O)₂—F group with a compound C of formula (I) R—O—SiR¹R²R³ in the presence of a catalyst D to form a mixture of products comprising said lithium salt A comprising at least one group —S(O)₂—OR; said catalyst D being a compound with a pKa of greater than 11 measured at 25° C.

2. The process of claim 1, characterized in that the substituent R of said compound C comprises at least one carbon-carbon double bond.

3. The process of claim 1, characterized in that said lithium salt B is of formula LiN(S(O)₂—F)(S(O)₂R′) and said lithium salt A is of formula (IV) LiN(S(O)₂—OR)(S(O)₂R′) in which R′ is a substituent selected from the group consisting of F, C₁-C₄ alkyl, C₁-C₄ perfluoroalkyl, C₁-C₄ alkoxy and C₁-C₄ perfluoroalkoxy.

4. The process of claim 1, characterized in that said lithium salt B comprises two —S(O)₂—F groups to form a lithium salt A comprising two groups —S(O)₂—OR.

5. The process of claim 1, characterized in that said lithium salt B is Li—N(S(O)₂—F)₂ and said lithium salt A is of formula (III) Li—N(S(O)₂—OR)₂.

6. The process of claim 1, characterized in that said compound C is of formula (I) R—O—SiR¹R²R³ in which the groups R′, R² and R³ are, independently of each other, selected from the group consisting of H and C₁-C₁₀ alkyl.

7. The process of claim 1, characterized in that said compound C is of formula (I) R—O—SiR¹R²R³ in which the group R is of formula R⁴—X—(Y)ₙ— in which R⁴ is a radical comprising at least one carbon-carbon double bond; X is a heteroatom selected from the group consisting of O, S and N(R⁵) in which R⁵ is H or C₁-C₁₀ alkyl; Y is a group selected from the group consisting of C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹) C(R⁸)(R⁹)]— with R⁶ and R⁷ independently selected for each carbon atom and for each unit Y from H or F, R⁸ and R⁹ independently selected for each carbon atom and for each unit Y from H, F or CH₃, m is an integer from 1 to 50, z=O if X is O; n is an integer from 1 to 50.

8. The process of claim 1, characterized in that the group R⁴ is of formula (Ia), (Ib), (Ic) or (Id): 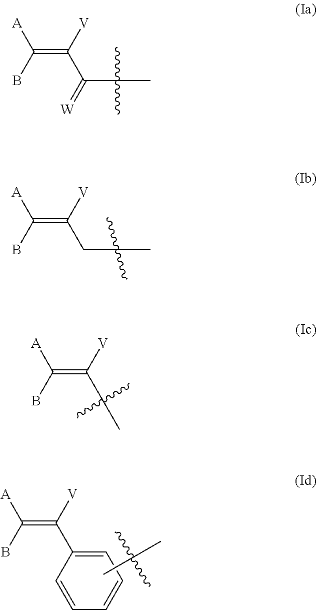
in which:
A, B and V are, independently of each other, selected from the group consisting of H, F, Cl, Br, I, C₁-C₁₀ alkyl optionally substituted with one or more substituents R″, C₂-C₁₀ alkenyl optionally substituted with one or more substituents R″, C₃-C₁₀ cycloalkyl optionally substituted with one or more substituents R″ or one or more C₁-C₅ alkyl groups, C₄-C₁₀ cycloalkenyl optionally substituted with one or more substituents R″ or one or more C₁-C₅ alkyl groups, C₆-C₁₂ aryl optionally substituted with one or more substituents R″ or one or more C₁-C₅ alkyl groups;
W is selected from O and S;
R″ is selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester.

9. The process of claim 1, characterized in that said catalyst D is selected from the group consisting of N,N-diisopropylethylamine, 1,8-diazabicyclo(5.4.0)undec-7-ene, 6-(dibutylamino)-1,8-diazabicyclo[5.4.0]undec-7-ene, polystyrene-bound 1,8-diazabicyclo[5.4.0]undec-7-ene, 1,5,7-triazabicyclo[4.4.0]dec-5-ene, 7-methyl-1,5,7-triazabicyclo[4.4.0]dec-5-ene, 1,1,3,3-tetramethylguanidine, 2-tert-butyl-1,1,3,3-tetramethylguanidine, polystyrene-bound 1,5,7-triazabicyclo[4.4.0]dec-5-ene, 1,4-diazabicyclo[2.2.2]octane, quinuclidine, 1,5-diazabicyclo(4.3.0)non-5-ene, 2,6-di-tert-butylpyridine, 2,8,9-trimethyl-2,5,8,9-tetraaza-1-phosphabicyclo[3.3.3]undecane, cyclodiphosphazane, lithium diisopropylamide, sodium diisopropylamide, potassium diisopropylamide, magnesium diisopropylamide, calcium diisopropylamide, rubidium diisopropylamide, lithium bis(trimethylsilyl)amide, sodium bis(trimethylsilyl)amide, potassium bis(trimethylsilyl)amide, magnesium bis(trimethylsilyl)amide, calcium bis(trimethylsilyl)amide, rubidium bis(trimethylsilyl)amide, lithium tetramethylpiperidide, sodium tetramethylpiperidide, potassium tetramethylpiperidide, magnesium tetramethylpiperidide, calcium tetramethylpiperidide, rubidium tetramethylpiperidide, [18-crown-6]—KHF₂, KHF₂, N,N′-diisopropylimidazonium, and bifluoride, tetrabutylammonium.

10. The process of claim 1 further comprising a step b) of purifying said lithium salt A; said step b) comprising extraction of said lithium salt A with a nonpolar organic solvent.

11. The process of claim 1, characterized in that step a) is performed at a temperature of from 10° C. to 50° C.

12. A lithium salt selected from the group consisting of formula (IIIa), (IIIb), (IIIc), (IIId), (IIIe), (IIIf), (IIIg), (IIIh), (IIIi), (IIIj), (IVa), (IVb), (IVc) and (IVd). 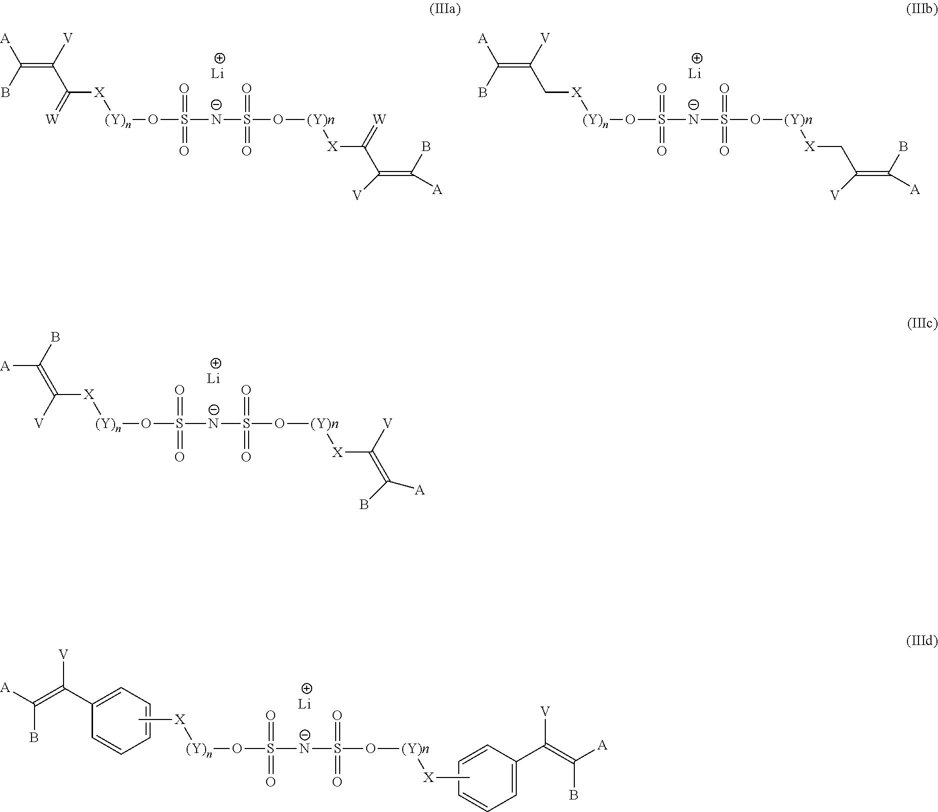 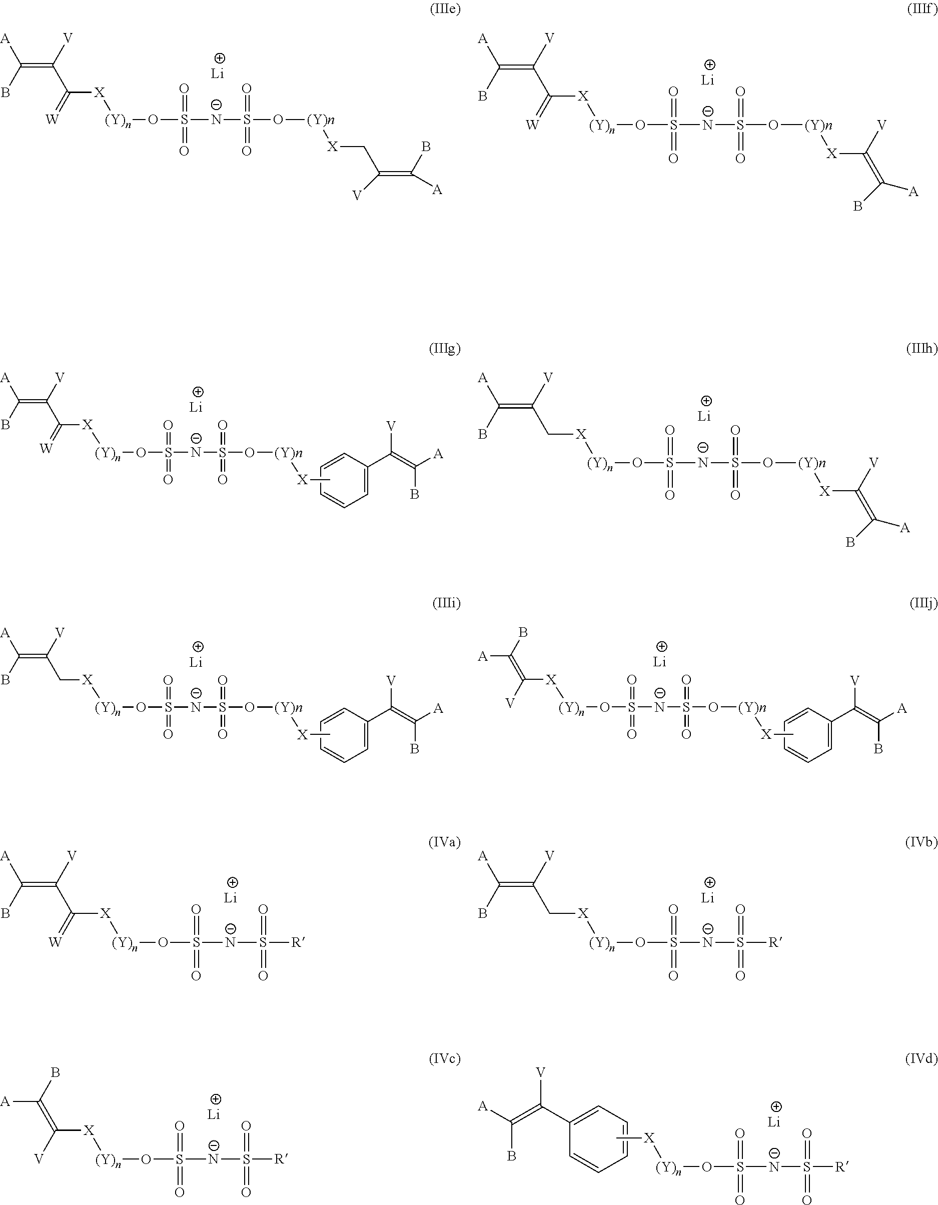
in which the substituents A, B, and V are independently of each other, selected from the group consisting of H, F, Cl, Br, I, C₁-C₁₀ alkyl optionally substituted with one or more substituents R″, C₂-C₁₀ alkenyl optionally substituted with one or more substituents R″, C₃-C₁₀ cycloalkyl optionally substituted with one or more substituents R″ or one or more C₁-C₅ alkyl groups, C₄-C₁₀ cycloalkenyl optionally substituted with one or more substituents R″ or one or more C₁-C₅ alkyl groups, C₆-C₁₂ aryl optionally substituted with one or more substituents R″ or one or more C₁-C₅ alkyl groups; R″ is selected from the group consisting of F, Cl, I, Br, C₁-C₄ ether, C₁-C₄ ester, C₁-C₄ thioether and C₁-C₄ thioester, preferably F, C₁-C₄ ether, C₁-C₄ ester, C₁-C₄ thioether and C₁-C₄ thioester;
W is selected from O and S;
X is a heteroatom selected from the group consisting of O, S and N(R⁵) in which R⁵ is H or C₁-C₁₀ alkyl;
Y is selected from the group consisting of C(R⁶)(R⁷)—, —C(R⁶)(R⁷)C(R⁶)(R⁷)SC(R⁶)(R⁷)— and —[(O)_{z}(C(R⁸)(R⁹)—C(R⁸)(R⁹)O)ₘ—C(R⁸)(R⁹) C(R⁸)(R⁹)]— with R⁶ and R⁷ independently selected for each carbon atom and for each unit Y from H or F, R⁸ and R⁹ independently selected for each carbon atom and for each unit Y from H, F or CH₃, m is an integer from 1 to 50, z=0 if X is O; n is an integer from 1 to 50; and
R′ is selected from the group consisting of F, C₁-C₄ alkyl, C₁-C₄ perfluoroalkyl, C₁-C₄ alkoxy and C₁-C₄ perfluoroalkoxy; preferably C₁-C₄ alkyl, C₁-C₄ perfluoroalkyl, C₁-C₄ alkoxy and C₁-C₄ perfluoroalkoxy.

13. The lithium salt of claim 12, selected from the group of formula (IIIa), (IIIb), (IIIc), (IIId), (IIIe), (IIIf), (IIIg), (IIIh), (IIIi), (IIIj), (IVa), (IVb), (IVc) and (IVd), in which
A and B are identical and are selected from the group consisting of H, F, Cl, Br and I;
V is selected from the group consisting of H, F, Cl, Br, I, C₁-C₁₀ alkyl optionally substituted with one or more substituents R″, C₂-C₁₀ alkenyl optionally substituted with one or more substituents R″, C₃-C₁₀ cycloalkyl optionally substituted with one or more substituents R″ or one or more C₁-C₅ alkyl groups, C₄-C₁₀ cycloalkenyl optionally substituted with one or more substituents R″ or one or more C₁-C₅ alkyl groups, C₆-C₁₂ aryl optionally substituted with one or more substituents R″ or one or more C₁-C₅ alkyl groups with R″ selected from the group consisting of F, Cl, I, Br, C₁-C₄ ether, C₁-C₄ ester, C₁-C₄ thioether, C₁-C₄ thioester;
W is O;
X is O;
Y is a group selected from the group consisting of CH₂CH₂, CH₂CF₂, CF₂CF₂, CH₂CHF, CF₂CH₂ and CHFCH₂, and n is an integer from 1 to 25; or
Y is a group —[(C(R⁸)(R⁹)—C(R⁸)(R⁹)O)ₘ—C(R⁸)(R⁹)C(R⁸)(R⁹)]— with R⁸ and R⁹ independently selected for each carbon atom and for each unit Y from H, F or CH₃, m is an integer from 1 to 25 and n is 1;
R′ is a substituent selected from the group consisting of F, C₁-C₄ alkyl, C₁-C₄ perfluoroalkyl, C₁-C₄ alkoxy and C₁-C₄ perfluoroalkoxy.

14. The lithium salt of claim 12, selected from the group of formula (IIIa), (IIIb), (IIIc), (IIId), (IIIe), (IIIf), (IIIg), (IIIh), (IIIi), (IIIj), (IVa), (IVb), (IVc) and (IVd)_{z} in which A and B are H, V is CH₃, W is O, X is O and Y is a group selected from the group consisting of CH₂CH₂, CH₂CF₂, CF₂CF₂, CH₂CHF, CF₂CH₂ and CHFCH₂, and n is an integer from 1 to 10; or Y is a group —[(C(R⁸)(R⁹)—C(R⁸)(R⁹)O)ₘ—C(R⁸)(R⁹)C(R⁸)(R⁹)]— with R⁸ and R⁹ independently selected for each carbon atom and for each unit Y from H, F or CH₃, m is an integer from 1 to 25 and n is 1;
R′ is a substituent selected from group consisting of F, C₁-C₄ alkyl, C₁-C₄ perfluoroalkyl, C₁-C₄ alkoxy and C₁-C₄ perfluoroalkoxy.

15. The lithium salt of formula (IIIa) of claim 12, in which A and B are H, V is CH₃, W is O, X is O and Y is a group selected from the group consisting of CH₂CH₂, CH₂CF₂, CF₂CF₂, CH₂CHF, CF₂CH₂ and CHFCH₂, and n is an integer from 1 to 10; or
Y is a group —[(C(R⁸)(R⁹)—C(R⁸)(R⁹)O)ₘ—C(R⁸)(R⁹)C(R⁸)(R⁹)]— with R⁸ and R⁹ independently selected for each carbon atom and for each unit Y from H, F or CH₃, m is an integer from 1 to 25 and n is 1.

16. A polymer comprising monomer units derived from at least one lithium salt of claim 12.

17. The polymer of claim 16, characterized in that it also comprises monomer units resulting from a monomer M1 of formula R¹R²C=C(R³)C(O)R in which the substituents R¹, R² and R³ are selected, independently of each other, from the group consisting of H and C₁-C₅ alkyl; R is selected from the group consisting of —NHC(CH₃)₂CH₂C(O)CH₃ or —OR′ with R′ selected from the group consisting of H and C₁-C₁₈ alkyl optionally substituted with one or more —OH groups or a 5- or 10-membered heterocycle comprising at least one nitrogen atom in its cyclic chain.

18. The polymer of claim 16, characterized in that it also comprises monomer units resulting from a monomer M2 selected from the group consisting of vinylidene fluoride, vinyl fluoride, trifluoroethylene, chlorotrifluoroethylene, 1,2-difluoroethylene, tetrafluoroethylene, hexafluoropropylene, perfluoro(alkyl vinyl) ethers, perfluoro(1,3-dioxole), perfluoro(2,2-dimethyl-1,3-dioxole), the monomer of formula CF₂=CFOCF₂CF(CF₃)OCF₂CF₂X in which X is SO₂F, CO₂H, CH₂OH, CH₂OCN or CH₂OPO₃H, the monomer of formula CF₂=CFOCF₂CF₂SO₂F, the monomer of formula F(CF₂)nCH₂OCF=CF₂ in which n is 1, 2, 3, 4 or 5, the monomer of formula R¹CH₂OCF=CF₂ in which R′ is hydrogen or F(CF₂)m and m has the value 1, 2, 3 or 4, the monomer of formula R²OCF=CH₂ in which R² is F(CF₂)p and p is 1, 2, 3 or 4, perfluorobutylethylene, trifluoropropene, tetrafluoropropene, hexafluoroisobutylene, perfluorobutylethylene, pentafluoropropene, bromotrifluoroethylene, chlorofluoroethylene, chlorotrifluoropropene and 2-trifluoromethyl-3,3,3-trifluoro-1-propene.

19. A binder for an electrode or a coating for a separator comprising the polymer of claim 16.

20. An electrode or separator comprising the polymer of claim 16.

21. A battery comprising said electrode or said separator of claim 20.
